(19) United States
(12) Patent Application Publication
(10) Pub. No.: US 2026/0152785 A1
(43) Pub. Date: Jun. 4, 2026

(54) Method of Detecting Nucleic Acid Analytes Using Dual-Specificity Primers

(71) Applicant: Gen-Probe Incorporated, San Diego, CA (US)

(72) Inventors: Charles B. Cappellari; Paul M. Darby, San Diego, CA (US)

(73) Assignee: Gen-Probe Incorporated, San Diego, CA (US)

(21) Appl. No.: 19/109,506

(22) PCT Filed: Sep. 7, 2023
(86) PCT No.: PCT/US2023/073660

§ 371 Date: Mar. 6, 2025

Related U.S. Application Data
(60) Provisional application No. 63/375,009, filed on Sep. 8, 2022.

(51) Int. Cl. C12Q 1/6832; C12Q 1/6806; C12Q 1/6825; C12Q 1/6851; C12Q 1/6876

(52) U.S. Cl. CPC C12Q 1/6832; C12Q 1/6806; C12Q 1/6825; C12Q 1/6851; C12Q 1/6876

(57) ABSTRACT

Provided are compositions, kits, and methods that can be used to selectively amplify a first target nucleic acid that is distinguishable from a second target nucleic acid, where the two target nucleic acids have both common and different sequence domains. Structured primers useful in the amplification method include 5′ and 3′ target-hybridizing sequences separated from each other by a joining region that does not hybridize to the target nucleic acid. Polymerase-based extension of the structured primer depends on specific hybridization of the 5′ sequence of the primer, thereby facilitating hybridization and extension of the 3′ sequence of the primer.

### RELATED APPLICATIONS

This application claims the benefit of U.S. Provisional Application No. 63/375,009, filed Sep. 8, 2022. The entire disclosure of this prior application is hereby incorporated by reference.

### REFERENCE TO ELECTRONIC SEQUENCE LISTING

The application contains a Sequence Listing which has been submitted electronically in .XML format and is hereby incorporated by reference in its entirety. Said .XML copy, created on Jan. 26, 2026, is named “01159-0057-00US.xml” and is 18,910 bytes in size. The sequence listing contained in this .XML file is part of the specification and is hereby incorporated by reference herein in its entirety.

### FIELD

This disclosure generally relates to the field of biotechnology. More specifically, the disclosure relates to compositions, kits, and methods for detecting the juxtaposition or joining of nucleic acid sequences, as may be associated with gene rearrangements, spliced or processed RNA, insertions into chromosomal nucleic acids, and the like. Still more specifically, the disclosure relates to a technique that preferentially amplifies one target nucleic acid in an amplification reaction that includes a related target nucleic acid, where the two nucleic acids share a substantially similar sequence in common.

### BACKGROUND

Detecting nucleic acid analytes having two juxtaposed nucleic acid sequences has important applications in the field of molecular diagnostics, including assays based on genomic structure or alternative RNA processing. For example, U.S. Pat. No. 8,551,699 discloses that joining of exon 3 to exon 4a is characteristic of substantially all alternative splice products of the over-expressed PCA3 transcript in prostate cancer cells. Since this RNA splice event does not occur in cells associated with benign prostatic hyperplasia, detection of this splice junction in RNA from prostate cells can be used for diagnosing prostate cancer. In another application relevant to alternative RNA processing, formation of a splice junction between the Cμ4 constant region sequence and exon sequences encoding a membrane anchor of the IgM antibody can be used for assessing the effects of drug treatment on RNA processing in a model system for the syndrome known as “common variable immunodeficiency” (see Sherr et al., J. Exp. Med., 168:55-71 (1988)). Yet other applications involve assessment of nucleic acid junctions at the genome level.

In some instances, juxtaposed sequences present in chromosomal DNA can indicate the presence of infectious agents, such as drug-resistant bacteria. For example, Staphylococcus aureus is a coagulase-positive opportunistic pathogen that causes bacteremia, pneumonia, abscesses, osteomyelitis, and infections of the heart (endocarditis, myocarditis, and pericarditis) and central nervous system (cerbritis and meningitis), and more. A methicillin-resistant strain of Staphylococcus aureus (“MRSA”) often requires treatment with more costly and/or toxic antibiotics typically considered a last line of defense. MRSA carries a mecA or mecC gene, which encode a β-lactam-resistant penicillin-binding protein that confers resistance to β-lactam antibiotics including but not limited to methicillin and penicillin. The mecA and mecC genes are located within a mobile genetic element termed SCCmec which also generally contains terminal inverted and direct repeats and a set of site-specific recombinase genes (ccrA, ccrB, and ccrC) that catalyze integration of SCCmec into the orfX gene on the S. aureus chromosome, thereby transforming methicillin-sensitive S. aureus (“MSSA”) into MRSA (Ito et al., 1999, Antimicrob. Agents Chemother. 43:1449-1458; Katayama et al., 2000, Antimicrob. Agents Chemother. 44:1549-1555; Huletsky et al., US Patent Pub. No. 2008/0227087). This integration event creates a junction that joins bacterial chromosomal sequences and sequences of the mobile genetic element encoding the drug resistance marker. Thus, the junction between SCCmec sequences and orfX sequences is a characteristic of MRSA nucleic acid, but not of MSSA nucleic acid. Notably, MRSA can spread easily in community and hospital settings, including through surface contact.

Generally speaking, there is a need for compositions and methods that can provide rapid and accurate detection of juxtaposed nucleic acid sequences, including detection of nucleic acid junctions resulting from alternative RNA processing, gene rearrangements, or the presence of chromosomal insertion sequences. As a specific example, there is a need for compositions and methods useful for identifying one or more of the various types of MRSA and/or discriminating them from MSSA and methicillin-resistant coagulase-negative staphylococcus (CNS). The present disclosure addresses these needs.

### SUMMARY

Provided herein are the following embodiments.

Embodiment 1 is a method of determining whether a test sample comprises a first nucleic acid without detecting a second nucleic acid, wherein the first and second nucleic acids comprise a common first sequence, and wherein the first and second nucleic acids have different sequences downstream from the common first sequence (preferably located upstream of the different downstream sequences), the method comprising the steps of: (a) contacting the test sample with a dual-specificity primer that comprises (i) a 5′ sequence of nucleotides that stably hybridizes to the downstream sequence of the first nucleic acid without stably hybridizing to the downstream sequence of the second nucleic acid, (ii) a 3′ sequence of nucleotides that is complementary to the common first sequence, wherein 3′ sequence of nucleotides hybridizes to the common first sequence only when 5′ sequence of nucleotides is stably hybridized to the downstream sequence of the first nucleic acid, (iii) a joining region between 5′ sequence of nucleotides and 3′ sequence of nucleotides, and (iv) a template termination moiety positioned between 5′ sequence of nucleotides and the joining region, wherein the joining region does not stably hybridize to any nucleic acid sequence that separates the common first sequence from either the downstream sequence of the first nucleic acid or the downstream sequence of the second nucleic acid; (b) performing an amplification reaction, wherein the amplification reaction comprises the dual-specificity primer and nucleic acids of the test sample; (c) detecting formation of any of an amplicon comprising 3′ sequence of nucleotides or the complement thereof that may have been produced in the amplification reaction; and (d) determining either that (i) the test sample comprises the first nucleic acid if the amplicon is detected in step (c), or (ii) the test sample does not comprise the first nucleic acid if the amplicon is not detected in step (c).

Embodiment 2 is the method of embodiment 1, wherein step (a) comprises combining in a reaction vessel each of the test sample, the dual-specificity primer, a pH buffer, deoxyribonucleotide triphosphates, and at least one polymerase to form an amplification reaction mixture.

Embodiment 3 is the method of either embodiment 1 or embodiment 2, wherein each of steps (a) to (d) is carried out using an automated nucleic acid analyzer that comprises each of: a temperature-regulated block or incubator that holds the reaction vessel and controls the temperature of the amplification reaction mixture therein, a fluorometer in optical communication with the amplification reaction mixture contained in the reaction vessel, and a computer that receives signals from the fluorometer.

Embodiment 4 is the method of embodiment 3, wherein the automated nucleic acid analyzer further performs a step, before step (a), of isolating nucleic acid from biological material that may be included in the test sample.

Embodiment 5 is the method of embodiment 4, wherein the step of isolating nucleic acid comprises capturing nucleic acid onto a solid support, washing the solid support to remove any material that was not captured, and retaining the solid support with the nucleic acid captured thereon.

Embodiment 6 is the method of either embodiment 1 or embodiment 2, wherein step (c) comprises detecting with an optical sensor.

Embodiment 7 is the method of embodiment 6, wherein the optical sensor comprises a fluorometer in optical communication with the amplification reaction mixture.

Embodiment 8 is the method of any one of embodiments 1 to 7, wherein the amplification reaction in step (b) comprises a PCR reaction, and wherein the amplicon in step (c) comprises DNA.

Embodiment 9 is the method of embodiment 8, wherein the PCR reaction in step (c) is a real-time PCR reaction with formation of any of the amplicon being detected as the amplification reaction is occurring.

Embodiment 10 is the method of any one of embodiments 1 to 9, wherein step (c) comprises detecting as the amplification reaction is occurring, and further comprises determining a threshold cycle value indicating a predetermined level of progress in the amplification reaction.

Embodiment 11 is the method of embodiment 10, further comprising after step (c), a step for quantifying any of the first nucleic acid in the test sample by comparing the threshold cycle value to a calibration curve.

Embodiment 12 is the method of any one of embodiments 1 to 11, wherein step (c) comprises detecting with a hybridization probe, wherein the hybridization probe comprises a detectable label.

Embodiment 13 is the method of embodiment 12, wherein the detectable label comprises a fluorescent moiety.

Embodiment 14 is the method of any one of embodiments 1 to 13, wherein the template termination moiety is positioned between 5′ sequence of nucleotides and the joining region to fully separate one from the other, and wherein the template termination moiety of the dual-specificity primer comprises a non-nucleotide moiety.

Embodiment 15 is the method of embodiment 14, wherein the non-nucleotide moiety comprises at least one glycol linkage.

Embodiment 16 is the method of any one of embodiments 1 to 15, wherein 5′ sequence of nucleotides of the dual-specificity primer is 10 to 60 nucleotides in length.

Embodiment 17 is the method of any one of embodiments 1 to 16, wherein 3′ sequence of nucleotides of the dual-specificity primer is 6 to 10 nucleotides in length.

Embodiment 18 is the method of any one of embodiments 1 to 17, wherein the joining region of the dual-specificity primer is 5 to 50 nucleotides in length.

Embodiment 19 is the method of embodiment 18, wherein the joining region of the dual-specificity primer is of a length that is the same as the number of nucleotides separating sequences in the first nucleic acid that are complementary to 5′ sequence of nucleotides and 3′ sequence of nucleotides of the dual-specificity primer.

Embodiment 20 is the method of embodiment 18, wherein the joining region of the dual-specificity primer is of a length that is greater than the number of nucleotides separating sequences in the first nucleic acid that are complementary to 5′ sequence of nucleotides and 3′ sequence of nucleotides of the dual-specificity primer.

Embodiment 21 is the method of embodiment 18, wherein the joining region of the dual-specificity primer is of a length that is less than the number of nucleotides separating sequences in the first nucleic acid that are complementary to 5′ sequence of nucleotides and 3′ sequence of nucleotides of the dual-specificity primer.

Embodiment 22 is the method of any one of embodiments 1 to 21, wherein 5′ sequence of nucleotides and 3′ sequence of nucleotides have a combined length of 17 to 29 nucleotides.

Embodiment 23 is the method of embodiment 22, wherein 5′ sequence of nucleotides is 11 to 23 nucleotides long.

Embodiment 24 is the method of any one of embodiments 2 to 23, wherein the amplification reaction mixture further comprises an amplification oligonucleotide that hybridizes to an extension product of the dual-specificity primer when using the first nucleic acid as a template.

Embodiment 25 is the method of embodiment 24, wherein the amplification oligonucleotide is a primer having a 3′ hydroxyl moiety, and wherein the primer having the 3′ hydroxyl moiety is not a dual-specificity primer.

Embodiment 26 is the method of any one of embodiments 1 to 25, wherein step (d) is automated by a computer programmed with software.

Embodiment 27 is the method of any one of embodiments 1 to 26, wherein the amplification reaction further comprises an internal control nucleic acid that amplifies to produce an internal control amplicon even in the absence of the first and second nucleic acids.

Embodiment 28 is the method of any one of embodiments 1 to 27, wherein determining in step (d) that the test sample comprises the first nucleic acid is an indication that either a chromosomal rearrangement or a post-transcriptional RNA processing event occurred to join together the common first sequence and the downstream sequence of the first nucleic acid.

Embodiment 29 is the method of any one of embodiments 1 to 27, wherein the first nucleic acid is present in a first bacterial strain but not in a second bacterial strain, and wherein the second nucleic acid is present in the second bacterial strain but not in the first bacterial strain.

Embodiment 30 is the method of any one of embodiments 1 to 29, wherein the test sample comprises both of the first and second nucleic acids.

Embodiment 31 is a dual-specificity primer for amplifying a first nucleic acid without amplifying a second nucleic acid in the same nucleic acid amplification reaction, wherein the first and second nucleic acids comprise a common first sequence, and wherein the first and second nucleic acids have different sequences downstream from the common first sequence, the dual-specificity primer comprising: (i) a 5′ sequence of nucleotides that stably hybridizes to the downstream sequence of the first nucleic acid without stably hybridizing to the downstream sequence of the second nucleic acid, (ii) a 3′ sequence of nucleotides that is complementary to the common first sequence, wherein 3′ sequence of nucleotides hybridizes to the common first sequence only when 5′ sequence of nucleotides is stably hybridized to the downstream sequence of the first nucleic acid, (iii) a joining region between the 5′ sequence of nucleotides and 3′ sequence of nucleotides, and (iv) a template termination moiety positioned between 5′ sequence of nucleotides and the joining region, wherein the joining region does not stably hybridize to any nucleic acid sequence that separates the common first sequence from either the downstream sequence of the first nucleic acid or the downstream sequence of the second nucleic acid.

Embodiment 32 is the dual-specificity primer of embodiment 31, wherein the template termination moiety is positioned between 5′ sequence of nucleotides and the joining region to fully separate one from the other, and wherein the template termination moiety of the dual-specificity primer comprises a non-nucleotide moiety.

Embodiment 33 is the dual-specificity primer of embodiment 32, wherein the non-nucleotide moiety comprises at least one glycol linkage.

Embodiment 34 is the dual-specificity primer of any one of embodiments 31 to 33, wherein 5′ sequence of nucleotides is 10 to 60 nucleotides in length.

Embodiment 35 is the dual-specificity primer of any one of embodiments 31 to 34, wherein 3′ sequence of nucleotides is 6 to 10 nucleotides in length.

Embodiment 36 is the dual-specificity primer of any one of embodiments 31 to 35, wherein the joining region is 5 to 50 nucleotides in length.

Embodiment 37 is the dual-specificity primer of embodiment 36, wherein the joining region is of a length that is the same as the number of nucleotides separating sequences in the first nucleic acid that are complementary to 5′ sequence of nucleotides and 3′ sequence of nucleotides of the dual-specificity primer.

Embodiment 38 is the dual-specificity primer of embodiment 36, wherein the joining region of the dual-specificity primer is of a length that is greater than the number of nucleotides separating sequences in the first nucleic acid that are complementary to 5′ sequence of nucleotides and 3′ sequence of nucleotides of the dual-specificity primer.

Embodiment 39 is the dual-specificity primer of embodiment 36, wherein the joining region of the dual-specificity primer is of a length that is less than the number of nucleotides separating sequences in the first nucleic acid that are complementary to 5′ sequence of nucleotides and 3′ sequence of nucleotides of the dual-specificity primer.

Embodiment 40 is the dual-specificity primer of any one of embodiments 31 to 39, wherein 5′ sequence of nucleotides and 3′ sequence of nucleotides have a combined length of 17 to 29 nucleotides.

Embodiment 41 is the dual-specificity primer of embodiment 40, wherein 5′ sequence of nucleotides is 11 to 23 nucleotides long.

Embodiment 42 is the dual-specificity primer of any one of embodiments 31 to 41, wherein 5′ sequence of nucleotides, 3′ sequence of nucleotides, and the joining region each consist of DNA.

Embodiment 43 is the dual-specificity primer of any one of embodiments 31 to 42, wherein the first nucleic acid is the product of either a chromosomal rearrangement or a post-transcriptional RNA processing event that joined together the common first sequence and the downstream sequence of the first nucleic acid.

Embodiment 44 is the method of any one of embodiments 31 to 42, wherein the first nucleic acid is present in a first bacterial strain but not in a second bacterial strain, and wherein the second nucleic acid is present in the second bacterial strain but not in the first bacterial strain.

Embodiment 45 is a kit containing one or more reagents for amplifying a first nucleic acid without amplifying a second nucleic acid in the same nucleic acid amplification reaction, wherein the first and second nucleic acids comprise a common first sequence, and wherein the first and second nucleic acids have different sequences downstream from the common first sequence, the kit comprising, in one or more vials: a dual-specificity primer that comprises (i) a 5′ sequence of nucleotides that stably hybridizes to the downstream sequence of the first nucleic acid without stably hybridizing to the downstream sequence of the second nucleic acid, (ii) a 3′ sequence of nucleotides that is complementary to the common first sequence, wherein the 3′ sequence of nucleotides hybridizes to the common first sequence only when 5′ sequence of nucleotides is stably hybridized to the downstream sequence of the first nucleic acid, (iii) a joining region between 5′ sequence of nucleotides and 3′ sequence of nucleotides, and (iv) a template termination moiety positioned between 5′ sequence of nucleotides and the joining region, wherein the joining region does not stably hybridize to any nucleic acid sequence that separates the common first sequence from either the downstream sequence of the first nucleic acid or the downstream sequence of the second nucleic acid; and an amplification oligomer, the amplification oligomer being substantially complementary to a polymerase-dependent extension product of the dual-specificity primer using the first nucleic acid as a template, wherein the dual-specificity primer and the amplification oligomer are configured to produce a first nucleic acid amplicon when used in combination with the first nucleic acid as the template in a nucleic acid amplification reaction.

Embodiment 46 is the kit of embodiment 45, wherein the amplification oligomer is a second primer.

Embodiment 47 is the kit of embodiment 46, wherein the second primer does not comprise a joining region or a template termination moiety.

Embodiment 48 is the kit of any one of embodiments 45 to 47, further comprising a pH buffer, a plurality of deoxyribonucleotide triphosphates (dNTPs), and at least one polymerase.

Embodiment 49 is the kit of embodiment 48, wherein the at least one polymerase comprises at least one thermostable polymerase.

Embodiment 50 is the kit of embodiment 48, further comprising a hybridization probe with a sequence substantially complementary to a sequence contained within the first nucleic acid amplicon, the hybridization probe comprising a detectable label.

Embodiment 51 is the kit of embodiment 50, wherein the detectable label of the hybridization probe comprises a fluorescent label.

Embodiment 52 is the kit of any one of embodiments 45 to 51, further comprising (i) a control template that is amplifiable in a nucleic acid amplification reaction using the dual-specificity primer and the amplification oligomer to produce a control amplicon, and (ii) a control hybridization probe with a sequence substantially complementary to a sequence contained within the control amplicon but not within the first nucleic acid amplicon, the control hybridization probe comprising a detectable label.

Embodiment 53 is the kit of embodiment 52, wherein the detectable label of the control hybridization probe comprises a fluorescent label.

Embodiment 54 is the kit of any one of embodiments 45 to 53, wherein the template termination moiety is positioned between 5′ sequence of nucleotides and the joining region to fully separate one from the other, and wherein the template termination moiety of the dual-specificity primer comprises a non-nucleotide moiety.

Embodiment 55 is the kit of embodiment 54, wherein the non-nucleotide moiety comprises at least one glycol linkage.

Embodiment 56 is the kit of any one of embodiments 45 to 55, wherein 5′ sequence of nucleotides of the dual-specificity primer is 10 to 60 nucleotides in length.

Embodiment 57 is the kit of any one of embodiments 45 to 56, wherein 3′ sequence of nucleotides of the dual-specificity primer is 6 to 10 nucleotides in length.

Embodiment 58 is the kit of any one of embodiments 45 to 57, wherein the joining region of the dual-specificity primer is 5 to 50 nucleotides in length.

Embodiment 59 is the kit of embodiment 58, wherein the joining region of the dual-specificity primer is of a length that is the same as the number of nucleotides separating sequences in the first nucleic acid that are complementary to 5′ sequence of nucleotides and 3′ sequence of nucleotides of the dual-specificity primer.

Embodiment 60 is the kit of embodiment 58, wherein the joining region of the dual-specificity primer is of a length that is greater than the number of nucleotides separating sequences in the first nucleic acid that are complementary to 5′ sequence of nucleotides and 3′ sequence of nucleotides of the dual-specificity primer.

Embodiment 61 is the kit of embodiment 58, wherein the joining region of the dual-specificity primer is of a length that is less than the number of nucleotides separating sequences in the first nucleic acid that are complementary to 5′ sequence of nucleotides and 3′ sequence of nucleotides of the dual-specificity primer.

Embodiment 62 is the kit of any one of embodiments 45 to 61, wherein 5′ sequence of nucleotides and 3′ sequence of nucleotides of the dual-specificity primer have a combined length of 17 to 29 nucleotides.

Embodiment 63 is the kit of embodiment 62, wherein 5′ sequence of nucleotides of the dual-specificity primer is 11 to 23 nucleotides long.

Embodiment 64 is the kit of any one of embodiments 45 to 63, wherein 5′ sequence of nucleotides, 3′ sequence of nucleotides, and the joining region of the dual-specificity primer consist of DNA.

Embodiment 65 is the kit of any one of embodiments 45 to 64, wherein the joining region of the dual-specificity primer comprises a phage T7 promoter.

Embodiment 66 is the kit of any one of embodiments 45 to 65, wherein the first nucleic acid is the product of either a chromosomal rearrangement or a post-transcriptional RNA processing event that joined together the common first sequence and the downstream sequence of the first nucleic acid.

Embodiment 67 is the kit of any one of embodiments 45 to 65, wherein the first nucleic acid is present in a first bacterial strain but not in a second bacterial strain, and wherein the second nucleic acid is present in the second bacterial strain but not in the first bacterial strain.

### BRIEF DESCRIPTION OF THE DRAWINGS

FIGS. 1A-1F are schematic diagrams illustrating the structure and function of dual-specificity primers. FIG. 1A schematically represents hybridization of a dual-specificity primer to a template. FIG. 1B schematically represents polymerase-based extension of the hybridized dual-specificity primer. FIG. 1C schematically represents the dual-specificity primer extension product (produced in FIG. 1B) hybridized to an opposite-strand primer. The opposite-strand primer is shown as a “standard” primer that lacks a template termination moiety. FIG. 1D schematically represents extension of the opposite-strand primer shown hybridized in FIG. 1C. FIG. 1E schematically represents hybridization of a dual-specificity primer to the extension product of the opposite-strand primer from FIG. 1D. FIG. 1F schematically represents extension of the dual-specificity primer of FIG. 1E.

FIG. 2 shows the sequences of a standard primer and two dual-specificity primers discussed in Example 1, and their alignment with the relevant template sequence. In order from top to bottom, aligned sequences correspond to: PRIMER 3 (SEQ ID NOS: 3 and 15), PRIMER 2 (SEQ ID NOS: 2 and 14), PRIMER 1 (SEQ ID NO: 1), SEQ ID NO:12, and SEQ ID NO: 11. Note that the upper three sequences appear in 3′ to 5′ orientation to demonstrate complementarity to SEQ ID NO: 12. Nucleotides in the primers of SPRIMERS 1-3 (top three entries) that are complementary to the MRSA template including SEQ ID NO:12 (entry four) are indicated by “\hat{ }” symbols. Nucleotides in the MSSA template including SEQ ID NO:11 that are identical to nucleotides at the same positions in SEQ ID NO:12 are indicated by “.” symbols. Dashes (heavy or light) are inserted into the alignment to maintain registration, but do not represent nucleotides. Filled markers in the dual-specificity primers of the first two entries (PRIMERS 3 and 2) represent template termination moieties.

FIGS. 3A-3F show real-time run curves for amplification reactions performed using the disclosed primers to amplify two different template nucleic acids. FIGS. 3A and 3B present results obtained using the standard reverse primer that lacks a template termination moiety of SEQ ID NO:1 to amplify the MSSA template including SEQ ID NO:11 (3A), or the MRSA template including SEQ ID NO:12 (3B). FIGS. 3C and 3D present results obtained using the dual-specificity primer of PRIMER 2 to amplify the MSSA template including SEQ ID NO: 11 (3C), or the MRSA template including SEQ ID NO:12 (3D). FIGS. 3E and 3F present results obtained using the dual-specificity primer of PRIMER 3 to amplify the MSSA template including SEQ ID NO:11 (3E), or the MRSA template including SEQ ID NO:12 (3F).

FIGS. 4A-4C show plots illustrating the linear nature of amplification profiles obtained using the disclosed primers in amplification reactions. FIG. 4A is a plot of results obtained using the MRSA template and the standard reverse primer of SEQ ID NO:1. FIG. 4B is a plot of results obtained using the MRSA template and the dual-specificity primer of PRIMER 2. FIG. 4C is a plot of results obtained using the dual-specificity primer of PRIMER 3 to amplify the MSSA template including SEQ ID NO:11 (∘), or the MRSA template including SEQ ID NO: 12 (Δ). All numerical data is taken from Table 1.

FIG. 5 schematically illustrates hybrid duplexes between the dual-specificity primers presented in Table 2 and the model MRSA target nucleic acid that includes a junction. The target nucleic acid sequence is presented in 5′ to 3′ orientation in each instance. Dual-specificity primers are shown with complementary base pairing, where the primer appears in the 3′ to 5′ orientation. The partial sequence of the MRSA target nucleic acid shown as the lower strand in the hybrid duplexes is given by SEQ ID NO:13. In order from top to bottom, dual-specificity primers shown in the duplexes have sequences given by PRIMER 2 (SEQ ID NOS: 2 and 14), PRIMER 4 (SEQ ID NOS: 4 and 14), PRIMER 5 (SEQ ID NOS: 5 and 14), PRIMER 6 (SEQ ID NOS: 6 and 14), and PRIMER 7 (SEQ ID NOS: 7 and 14). Boxed nucleotides indicate template sequences complementary to 3′ and 5′ sequences of nucleotides in the dual-specificity primer of PRIMER 2.

FIG. 6 presents calibration plots of the numerical results from Table 3. Five different dual-specificity primers illustrated in FIG. 5 were used to amplify target nucleic acids at different input copy levels (from about 500 copies/ml up to about 500,000 copies/ml). Primers used in the procedure were: PRIMER 2 (∘), PRIMER 4 (□), PRIMER 5 (Δ), PRIMER 6 (⋄), and PRIMER 7 (X).

### DETAILED DESCRIPTION OF CERTAIN EMBODIMENTS

### Definitions

Before describing the present teachings in detail, it is to be understood that the disclosure is not limited to specific compositions or process steps, as such may vary. It should be noted that, as used in this specification and the appended claims, the singular form “a,” “an,” and “the” include plural references, and expressions such as “one or more items” include singular references unless the context clearly dictates otherwise. Thus, for example, reference to “an oligomer” includes a plurality of oligomers and the like; in a further example, a statement that “one or more secondary detection oligomers are FRET cassettes” includes a situation in which there is exactly one secondary detection oligomer and it is a FRET cassette. The conjunction “or” is to be interpreted in the inclusive sense (i.e., as equivalent to “and/or”), unless the inclusive sense would be unreasonable in the context. When “at least one” member of a class (e.g., oligomer) is present, reference to “the” member (e.g., oligomer) refers to the present member (if only one) or at least one of the members (e.g., oligomers) present (if more than one).

It will be appreciated that there is an implied “about” prior to the temperatures, concentrations, times, etc., discussed in the present disclosure, such that slight and insubstantial deviations are within the scope of the present teachings herein. In general, the term “about” indicates insubstantial variation in a quantity of a component of a composition not having any significant effect on the activity or stability of the composition (e.g., within 10%, 5%, 2%, or 1%). Accordingly, unless indicated to the contrary, the numerical parameters set forth in the following specification and attached claims are approximations that may vary depending upon the desired properties sought to be obtained. At the very least, and not as an attempt to limit the application of the doctrine of equivalents to the scope of the claims, each numerical parameter should at least be construed considering the number of reported significant digits and by applying ordinary rounding techniques. All ranges are to be interpreted as encompassing the endpoints in the absence of express exclusions such as “not including the endpoints” and all points between the endpoints; thus, for example, “within 10-15” includes the values 10 and 15. One skilled in the art will understand that ranges recited herein include all whole and rational numbers within the range (e.g., 90%-100% includes 92% and 98.377%). It is to be understood that both the foregoing general description and detailed description are exemplary and explanatory only and are not restrictive of the teachings. To the extent that any material incorporated by reference is inconsistent with the express content of this disclosure, the express content controls.

A “sample” refers to material that may contain a target nucleic acid, including but not limited to biological, clinical, environmental, and food samples. Environmental samples include environmental material such as surface matter, soil, water and industrial samples, as well as samples obtained from food and dairy processing instruments, apparatus, equipment, utensils, disposable and non-disposable items. “Biological” or “clinical” samples refer to a tissue or material derived from a living or dead human or animal which may contain a target nucleic acid, including, for example, skin, wound, nasopharyngeal or throat swabs, nasal or bronchial washes, nasal aspirates, sputum, other respiratory tissue or exudates, biopsy tissue including lymph nodes, or body fluids such as blood or urine. A sample can be treated to physically or mechanically disrupt tissue or cell structure to release intracellular nucleic acids into a solution which may contain enzymes, buffers, salts, detergents and the like, to prepare the sample for analysis. These examples are not to be construed as limiting the sample types applicable to the present disclosure.

“Nucleic acid” and “polynucleotide” refer to a multimeric compound comprising nucleosides or nucleoside analogs which have nitrogenous heterocyclic bases or base analogs linked together to form a polynucleotide, including conventional RNA, DNA, mixed RNA-DNA, and polymers that are analogs thereof. A nucleic acid “backbone” can be made up of a variety of linkages, including one or more of sugar-phosphodiester linkages, peptide-nucleic acid bonds (“peptide nucleic acids” or PNA; International Pub. No. WO 95/32305), phosphorothioate linkages, methylphosphonate linkages, or combinations thereof. Sugar moieties of a nucleic acid can be ribose, deoxyribose, or similar compounds with substitutions (e.g., 2′ methoxy or 2′ halide substitutions). Nitrogenous bases can be conventional bases (A, G, C, T, U), analogs thereof (e.g., inosine, or others; see The Biochemistry of the Nucleic Acids 5-36, Adams et al., ed., 11^{th} ed., 1992), derivatives of purines or pyrimidines (e.g., N⁴-methyl deoxyguanosine, deaza- or aza-purines, deaza- or aza-pyrimidines, pyrimidine bases with substituent groups at the 5 or 6 position (e.g., 5-methylcytosine), purine bases with a substituent at the 2, 6, or 8 positions, 2-amino-6-methylaminopurine, O⁶-methylguanine, 4-thio-pyrimidines, 4-amino-pyrimidines, 4-dimethylhydrazine-pyrimidines, and O⁴-yl-pyrimidines; U.S. Pat. No. 5,378,825 and International Pub. No. WO 93/13121). Nucleic acids can include one or more “abasic” residues where the backbone includes no nitrogenous base for position(s) of the polymer (U.S. Pat. No. 5,585,481). A nucleic acid can comprise only conventional RNA or DNA sugars, bases and linkages, or can include both conventional components and substitutions (e.g., conventional bases with 2′ methoxy linkages, or polymers containing both conventional bases and one or more base analogs). Nucleic acid includes “locked nucleic acid” (LNA), an analogue containing one or more LNA nucleotide monomers with a bicyclic furanose unit locked in an RNA mimicking sugar conformation, which enhance hybridization affinity toward complementary RNA and DNA sequences (Vester and Wengel, 2004, Biochemistry 43 (42): 13233-41). Embodiments of oligomers that can affect stability of a hybridization complex include PNA oligomers, oligomers that include 2′-methoxy or 2′-fluoro substituted RNA, or oligomers that affect the overall charge, charge density, or steric associations of a hybridization complex, including oligomers that contain charged linkages (e.g., phosphorothioates) or neutral groups (e.g., methylphosphonates). Methylated cytosines such as 5-methylcytosines can be used in conjunction with any of the foregoing backbones/sugars/linkages including RNA or DNA backbones (or mixtures thereof) unless otherwise indicated. RNA and DNA equivalents have different sugar moieties (i.e., ribose versus deoxyribose) and can differ by the presence of uracil in RNA and thymine in DNA. The differences between RNA and DNA equivalents do not contribute to differences in homology because the equivalents have the same degree of complementarity to a particular sequence. It is understood that when referring to ranges for the length of an oligonucleotide, amplicon, or other nucleic acid, that the range is inclusive of all whole numbers (e.g., 19-25 contiguous nucleotides in length includes 19, 20, 21, 22, 23, 24, and 25).

“C residues” include methylated and unmethylated cytosines unless the context indicates otherwise. In some embodiments, methylated cytosines comprise or consist of 5-methylcytosines.

An “oligomer” or “oligonucleotide” refers to a nucleic acid of generally less than 1,000 nucleotides (nt), including those in a size range having a lower limit of about 2 to 5 nt and an upper limit of about 500 to 900 nt. Some particular embodiments are oligomers in a size range with a lower limit of about 5 to 15, 16, 17, 18, 19, or 20 nt and an upper limit of about 50 to 600 nt, and other particular embodiments are in a size range with a lower limit of about 10 to 20 nt and an upper limit of about 22 to 100 nt. Oligomers can be purified from naturally occurring sources, but can be synthesized by using any well known enzymatic or chemical method. Oligomers can be referred to by a functional name (e.g., capture probe, primer or promoter primer) but those skilled in the art will understand that such terms refer to oligomers. Oligomers can form secondary and tertiary structures by self-hybridizing or by hybridizing to other polynucleotides. Such structures can include, but are not limited to, duplexes, hairpins, cruciforms, bends, and triplexes. Oligomers may be generated in any manner, including chemical synthesis, DNA replication, reverse transcription, PCR, or a combination thereof. In some embodiments, oligomers that form invasive cleavage structures are generated in a reaction (e.g., by extension of a primer in an enzymatic extension reaction).

A “capture probe” is an oligonucleotide that can be joined, either directly or indirectly, to a solid support, where the capture probe includes a sequence complementary to a target nucleic acid molecule that is to be isolated or purified. The capture probe hybridizes to the target nucleic acid molecule and immobilizes it to the solid support as part of a molecular complex that includes the solid support, the capture probe, and the target nucleic acid molecule. The complex can easily be washed to remove non-bound materials while retaining the captured target nucleic acid molecule. Separating the washed complex that includes the solid support effectively purifies and isolates the target nucleic acid molecule. A target capture procedure employing capture probes is illustrated in U.S. Pat. No. 6,110,678.

“A nucleic acid sequence” contained in a target nucleic acid refers to the sequence of bases and/or base analogs in a nucleic acid. For example, the term “nucleic acid sequence” includes both the sequence of bases and/or base analogs in an original target nucleic acid in a sample and the sequence of bases and/or base analogs in an amplicon generated from a strand of the original target nucleic acid in the sample.

As used herein, a “nucleotide” is a subunit of a nucleic acid consisting of a phosphate group, a 5-carbon sugar, and a nitrogenous base (sometimes referred to as a “nucleobase”). The 5-carbon sugar found in RNA is ribose. In DNA, the 5-carbon sugar is 2′-deoxyribose. The term also includes analogs of such subunits, such as a methoxy group at the 2′ position of the ribose (also referred to herein as “2′-O-Me” or “2′-methoxy”). Nucleotides in accordance with the disclosure can include conventional nitrogenous bases, as well as base analogs.

By “amplicon” or “amplification product” is meant a nucleic acid molecule generated in a nucleic acid amplification reaction using a target nucleic acid as a template. These nucleic acid molecules may be either single-stranded or double-stranded molecules. An amplicon or amplification product contains a target nucleic acid sequence that can be of the same or opposite sense as the target nucleic acid. In some embodiments, an amplicon has a length of about 100-2000 nucleotides, about 100-1500 nucleotides, about 100-1000 nucleotides, about 100-800 nucleotides, about 100-700 nucleotides, about 100-600 nucleotides, about 100-500 nucleotides, or about 100-200 nucleotides.

An “amplification oligonucleotide” or “amplification oligomer” refers to an oligonucleotide that hybridizes to a target nucleic acid, or its complement, and participates in a nucleic acid amplification reaction, e.g., serving as a primer and/or promoter-primer. Particular amplification oligomers contain at least about 10 contiguous bases, and optionally at least 11, 12, 13, 14, 15, 16, 17, 18, 19, or 20 contiguous bases, that are complementary to a region of the target nucleic acid sequence or its complementary strand. The contiguous bases can be at least about 80%, at least about 90%, or completely complementary to the target sequence to which the amplification oligomer binds. In some embodiments, an amplification oligomer comprises an intervening linker or non-complementary sequence between two segments of complementary sequence. Particular amplification oligomers are about 10 to about 60 bases long and optionally can include modified nucleotides.

A “primer” refers to an oligomer that hybridizes to a template nucleic acid and has a 3′ end that can be extended from an available 3′ hydroxyl moiety by a polymerase enzyme. A primer can be optionally modified (e.g., by including a 5′ region that is non-complementary to the target sequence). Such modification can include functional additions, such as tags, promoters, or other sequences used or useful for manipulating or amplifying the primer or target oligonucleotide. A primer modified with a 5′ promoter sequence can be referred to as a “promoter-primer.” A person of ordinary skill in the art of molecular biology or biochemistry will understand that an oligomer that can function as a primer can be modified to include a 5′ promoter sequence and then function as a promoter-primer, and, similarly, any promoter-primer can serve as a primer with or without its 5′ promoter sequence.

“Nucleic acid amplification” (also “amplification” or “amplify” or “amplifying” or gramatical equivalents) refers to any in vitro procedure that produces multiple copies of a target nucleic acid sequence, or its complementary sequence, or fragments thereof (i.e., an amplified sequence containing less than the complete target nucleic acid). Examples of nucleic acid amplification procedures include transcription associated methods, such as transcription-mediated amplification (TMA), nucleic acid sequence-based amplification (NASBA) and others (e.g., U.S. Pat. Nos. 5,399,491, 5,554,516, 5,437,990, 5,130,238, 4,868,105, and 5,124,246), replicase-mediated amplification (e.g., U.S. Pat. No. 4,786,600), the polymerase chain reaction (PCR) (e.g., U.S. Pat. Nos. 4,683,195, 4,683,202, and 4,800,159), ligase chain reaction (LCR) (e.g., EP Pat. App. 0320308) and strand-displacement amplification (SDA) (e.g., U.S. Pat. No. 5,422,252). Replicase-mediated amplification uses self-replicating RNA molecules, and a replicase such as QB-replicase. PCR amplification uses DNA polymerase, primers, and thermal cycling steps to synthesize multiple copies of the two complementary strands of DNA or cDNA. LCR amplification uses at least four separate oligonucleotides to amplify a target and its complementary strand by using multiple cycles of hybridization, ligation, and denaturation. SDA uses a primer that contains a recognition site for a restriction endonuclease that will nick one strand of a hemimodified DNA duplex that includes the target sequence, followed by amplification in a series of primer extension and strand displacement steps. Particular embodiments use PCR or TMA, but it will be apparent to persons of ordinary skill in the art that oligomers disclosed herein can be readily used as primers in other amplification methods. “Exponential” nucleic acid amplification refers to an amplification procedure where each strand of an amplified sequence serves as a template for amplification by opposite-strand primers to produce full-length copies of each strand.

As used herein, a “control template” refers to one or more nucleic acid strands that participate in a template-dependent nucleic acid amplification reaction, where the amplification reaction serves to demonstrate operability of the amplification procedure. For example, a control template may be included in a reaction mixture for the purpose of demonstrating the integrity of all reagents and conditions used in the amplification reaction. Detection of an amplification product produced from the control template can validate negative results for detecting a second analyte nucleic acid in the same amplification reaction.

Transcription associated amplification uses a DNA polymerase, an RNA polymerase, deoxyribonucleoside triphosphates, ribonucleoside triphosphates, a promoter-containing oligonucleotide, and optionally can include other oligonucleotides, to ultimately produce multiple RNA transcripts from a nucleic acid template (described in detail in U.S. Pat. Nos. 4,868,105, 5,124,246, 5,130,238, 5,399,491, 5,437,990, and 5,554,516; and International Pub. Nos. WO 88/01302, WO 88/10315, WO 94/03472, and WO 95/03430). Methods that use Transcription Mediated Amplification, or “TMA” (i.e., a type of transcription associated amplification) are described in detail previously (U.S. Pat. Nos. 5,399,491 and 5,554,516).

In cyclic amplification methods that detect amplicons in real-time, the term “threshold cycle” (sometimes “Tcycle”) is a measure of the emergence time of a signal associated with amplification of target, and may, for example, be approximately 10× standard deviation of the normalized reporter signal. Once an amplification reaches the “threshold cycle,” generally there is considered to be a positive amplification product of a sequence to which the probe binds. The identity of the amplification product can then be determined through methods known to one of skill in the art, such as gel electrophoresis, nucleic acid sequencing, and other such well known methods.

“Detection oligomer” or “probe” (e.g., “hybridization probe”) refers to an oligomer that interacts with a target nucleic acid to form a detectable complex. Well known examples of detection probes include: fluorescently labeled hydrolysis probes (sometimes referred to as “TAQMAN®” probes), molecular beacons, molecular torches. Invasive cleavage probes are yet alternative probes. Examples here include invasive probes and primary probes. An “invasive probe” refers to an oligonucleotide that hybridizes to a target nucleic acid at a location near the region of hybridization between a primary probe and the target nucleic acid, where the invasive probe oligonucleotide comprises a portion (e.g., a chemical moiety, or nucleotide, whether complementary to that target or not) that overlaps with the region of hybridization between the primary probe oligonucleotide and the target nucleic acid. The “primary probe” for an invasive cleavage assay includes a target-specific region that hybridizes to the target nucleic acid, and further includes a “5′ flap” region that is not complementary to the target nucleic acid. In general, detection can either be direct (i.e., probe hybridized directly to the target) or indirect (i.e., involving an intermediate structure that links a detectable label or detectably labeled molecule, such as a FRET cassette, to the target). The target sequence of a probe generally refers to the specific sequence within a larger sequence to which the probe hybridizes specifically. A detection oligomer can include target-specific sequences and a non-target-complementary sequence. Such non-target-complementary sequences can include sequences which will confer a desired secondary or tertiary structure, such as a flap or hairpin structure, which can be used to facilitate detection and/or amplification (e.g., U.S. Pat. Nos. 5,118,801, 5,312,728, 6,835,542, 6,849,412, 5,846,717, 5,985,557, 5,994,069, 6,001,567, 6,913,881, 6,090,543, and 7,482,127; International Pub. Nos. WO 97/27214 and WO 98/42873; Lyamichev et al., Nat. Biotech., 17:292 (1999); and Hall et al., PNAS, USA, 97:8272 (2000)). Probes of a defined sequence can be produced by techniques known to those of ordinary skill in the art, such as by chemical synthesis, and by in vitro or in vivo expression from recombinant nucleic acid molecules.

By “probe system” is meant a plurality of detection oligomers or probes for detecting a target sequence. In some embodiments, a probe system comprises at least primary and secondary probes. In some embodiments, a primary probe comprises a target-hybridizing sequence and a non-target-complementary sequence. In some embodiments, a primary probe undergoes nucleolysis (e.g., cleavage, such as 5′-cleavage or endonucleolysis) upon hybridization to a target sequence in the presence of an appropriate nuclease, such as structure-specific nuclease (e.g., a cleavase or 5′-nuclease). In some embodiments, such nucleolysis results in liberation of a “flap” or cleavage fragment from the primary probe that interacts with the secondary probe. In some embodiments, the secondary probe comprises at least one label. In some embodiments, the secondary probe comprises at least a pair of labels, such as an interacting pair of labels (e.g., a FRET pair or a fluorophore and quencher). In some embodiments, interaction of the secondary probe with a liberated flap of the primary probe results in a detectable change in the emission properties of the second probe, for example, as discussed below with respect to INVADER® assays, FRET, and/or quenching. In some embodiments, a probe system comprises a primary probe and a secondary probe configured to interact with a liberated flap of the primary probe. For example, the primary probe can be cleaved to give a liberated flap sufficiently complementary to the secondary probe or a segment thereof to form a complex.

“Hybridization” and “hybridize” refer to a process where two completely or partially complementary nucleic acid strands come together under assay conditions in a parallel or antiparallel orientation to form a stable structure having a double-stranded region. The two constituent strands of this double-stranded structure, sometimes called a hybrid, are held together by hydrogen bonds. Although these hydrogen bonds most commonly form between nucleotides containing the bases adenine and thymine or uracil (A and T or U) or cytosine and guanine (C and G), base pairing can also form between bases which are not members of these “canonical” pairs. Non-canonical base pairing is well-known in the art. (See R. L. P. Adams et al., The Biochemistry of the Nucleic Acids (11th ed. 1992))

“Stably hybridize” and “stably bind” (and gramatical equivalents) mean that two sequences, such as a target sequence and a primer or probe sequence, hybridize or bind sufficiently strongly by complementary base pairing as to permit a reaction to take place. This can occur when the temperature of the hybridization reaction is below the melting temperature (“Tm”) of the hybrid duplex that includes nucleic acid strands with the two sequences. Here the temperature of hybridization can be 1° C., 2° C., 3° C., 5° C., 10° C., or even more below the Tm of the hybrid duplex to result in stable binding.

As used herein, the term “Tₘ” is used in reference to the “melting temperature” of a nucleic acid strand with respect to a complementary nucleic acid strand. The melting temperature of nucleic acid duplex is the temperature at which a population of double-stranded nucleic acid molecules becomes half-dissociated into single strands in a hybridization reaction mixture. Measuring the melting temperature of a labeled nucleic acid duplex typically comprises plotting fluorescence as a function of temperature to produce a melting curve that is characteristic of the dissociation of the duplex. When the negative first derivative of a melting curve is graphed as a function of temperature, the Tₘ is identifiable as a peak. (See Ririe, et al., Analytical Biochemistry 245:154-160 (1997)).

As used herein, “specific” means pertaining to only one (or to only a particularly indicated group), such as having a particular effect on only one (or on only a particularly indicated group), or affecting only one (or only a particularly indicated group) in a particular way. For example, a cleaved 5′ flap specific for a FRET cassette will be able to hybridize to that FRET cassette, form an invasive cleavage structure, and promote a cleavage reaction, but will not be able to hybridize to a different FRET cassette (e.g., a FRET cassette having a different 5′ flap-hybridizing sequence) to promote a cleavage reaction. In addition, “specific” may be used in relation to a combination of oligonucleotides, such as a set of amplification and detection oligonucleotides (e.g., amplification oligonucleotides may amplify multiple target sequences non-specifically but the detection oligonucleotides will only detect a specific amplified sequence, thus making the combination specific).

As used herein, the term “specifically hybridizes” means that under given hybridization conditions a probe or primer detectably hybridizes substantially only to its target sequence(s) in a sample comprising the target sequence(s) (i.e., there is little or no detectable hybridization to non-targeted sequences). Notably, for example in the case of various MREJ target sequences, an oligomer can be configured to specifically hybridize to one or a set of MREJ target sequences. In some embodiments, an amplification or detection probe oligomer can hybridize to its target nucleic acid to form stable oligomer: target hybrid, but not form a sufficient number of stable oligomer: non-target hybrids for amplification or detection as the case may be. Amplification and detection oligomers that specifically hybridize to a target nucleic acid are useful to amplify and detect target nucleic acids, but not non-targeted nucleic acids, especially non-targeted nucleic acids of phylogenetically closely related organisms. Thus, the oligomer hybridizes to target nucleic acid to a sufficiently greater extent than to non-target nucleic acid to enable one having ordinary skill in the art to accurately amplify and/or detect the presence (or absence) of nucleic acid derived from the specified target (e.g., MRSA) as appropriate. In general, reducing the degree of complementarity between an oligonucleotide sequence and its target sequence will decrease the degree or rate of hybridization of the oligonucleotide to its target region. However, the inclusion of one or more non-complementary nucleosides or nucleobases may facilitate the ability of an oligonucleotide to discriminate against non-targeted nucleic acid sequences.

Specific hybridization can be measured using techniques known in the art and described herein, such as in the examples provided below. In some embodiments, there is at least a 10-fold difference between target and non-target hybridization signals in a test sample, at least a 100-fold difference, or at least a 1,000-fold difference. In some embodiments, non-target hybridization signals in a test sample are no more than the background signal level.

“Detecting the formation of an amplicon” means that the threshold cycle is equal to or less than a specified number of cycles (e.g., 32 cycles) of an amplification reaction. “Detecting the absence of an amplification means that no amplfication products are detected above background or that the threshold cycle of an amplification reaction is greater than the specified number of cycles.

By “stringent hybridization conditions,” or “stringent conditions” is meant conditions permitting an oligomer to preferentially hybridize to a target nucleic acid (e.g., MRSA nucleic acid) and not to nucleic acid derived from a closely related non-targeted organism. While the definition of stringent hybridization conditions does not vary, the actual reaction environment that can be used for stringent hybridization may vary depending upon factors including the GC content and length of the oligomer, the degree of similarity between the oligomer sequence and sequences of targeted and non-targeted nucleic acids that may be present in the test sample. Hybridization conditions include the temperature and the composition of the hybridization reagents or solutions. Exemplary hybridization assay conditions for amplifying and/or detecting target nucleic acids derived from one or more strains of MRSA with the oligomers of the present disclosure correspond to a temperature of about 63° C. to about 67° C. or about 64° C. to about 66° C. when the salt concentration, such as a divalent salt (e.g., MgCl₂), is in the range of about 5-21 mM. Additional details of hybridization conditions are set forth in the Examples section. Other acceptable stringent hybridization conditions could be easily ascertained by those having ordinary skill in the art.

“Label” or “detectable label” refers to a moiety or compound joined directly or indirectly to a probe that is detected, or that leads to a detectable signal. Direct joining can use covalent bonds or non-covalent interactions (e.g., hydrogen bonding, hydrophobic or ionic interactions, and chelate or coordination complex formation) whereas indirect joining can use a bridging moiety or linker (e.g., via an antibody or additional oligonucleotide(s)), which amplify a detectable signal. Any detectable moiety can be used. Preferred labels include non-nucleotide labels, which can be, for example, a chemiluminescent label (e.g., an acridinium ester) or a fluorescent label (e.g., a fluorophore or fluorescent moiety) joined directly or indirectly to the structure of an oligonucleotide. Examples of useful detectable labels include a radionuclide, a ligand such as biotin or avidin, an enzyme, an enzyme substrate, a reactive group, a chromophore such as a dye or particle (e.g., latex or metal bead) that imparts a detectable color, luminescent compounds (e.g. bioluminescent, phosphorescent, or chemiluminescent compounds), and fluorescent compounds (i.e., fluorophore). Embodiments of fluorophores include those that absorb light (e.g., have a peak absorption wavelength) in the range of about 495 to 690 nm and emit light (e.g., have a peak emission wavelength) in the range of about 520 to 710 nm, which include those known as FAM™, TET™, HEX™, CAL FLUOR® (Orange or Red), CY®, and QUASAR™ compounds. Fluorophores can be used in combination with a quencher molecule that absorbs a fluorescent emission when in close proximity to that fluorophore. Such quenchers are well known in the art, and include BLACK HOLE QUENCHER® (or BHQ®), BLACKBERRY QUENCHER® (or BBQ-650®), ECLIPSE®, or TAMRA™ compounds. Particular embodiments of detectable labels include a “homogeneous detectable label” that is detectable in a homogeneous system in which bound labeled probe in a mixture exhibits a detectable change compared to unbound labeled probe, which allows the label to be detected without physically removing hybridized from unhybridized labeled probe (e.g., U.S. Pat. Nos. 5,283,174, 5,656,207, and 5,658,737). Exemplary homogeneous detectable labels include chemiluminescent compounds, including acridinium ester (“AE”) compounds, such as standard AE or AE derivatives which are well known (U.S. Pat. Nos. 5,656,207, 5,658,737, and 5,639,604). Methods of synthesizing labels, attaching labels to nucleic acid, and detecting signals from labels are well known (e.g., Sambrook et al., Molecular Cloning, A Laboratory Manual, 2^{nd} ed. (Cold Spring Harbor Laboratory Press, Cold Spring Harbor, NY, 1989) at Chapt. 10; U.S. Pat. Nos. 5,658,737, 5,656,207, 5,547,842, 5,283,174, and 4,581,333; and EP Pat. App. 0 747 706). Particular methods of linking an AE compound to a nucleic acid are known (e.g., U.S. Pat. Nos. 5,585,481 and 5,639,604, see column 10, line 6 to column 11, line 3, and Example 8). Particular AE labeling positions include a probe's internal or central region where a duplex with a target nucleic exhibits full complementarity; or even at 3′ or 5′ terminus. Other detectably labeled probes include FRET cassettes, TAQMAN® probes, molecular torches, and molecular beacons. FRET cassettes are discussed in detail below. TAQMAN® probes include donor and acceptor labels, where fluorescence is detected upon enzymatically degrading the probe during amplification to separate the fluorophore (e.g., donor) from the quencher (e.g., acceptor). Molecular torches and molecular beacons exist in open and closed configurations, where the closed configuration quenches emission from the fluorophore, and the open configuration separates the fluorophore from the quencher to allow a change in detectable fluorescent signal.

Sequences are “sufficiently complementary” if they allow stable hybridization of two nucleic acid sequences to each other (e.g., stable hybrids of probe and target sequences). That is, a “sufficiently complementary” sequence can hybridize to another sequence by hydrogen bonding between a subset series of complementary nucleotides using standard base pairing (e.g., G:C, A:T, or A:U), although the two sequences can contain one or more residues (including abasic positions) that are not complementary so long as the stable hybridization complex is not disrupted. Sufficiently complementary sequences can be at least about 80%, at least about 90%, or completely complementary in the sequences that hybridize together. Appropriate hybridization conditions are well known to those skilled in the art, and can be predicted based on sequence composition, or can be determined empirically by using routine testing (e.g., Sambrook et al., Molecular Cloning, A Laboratory Manual, 2^{nd} ed. at §§ 1.90-1.91, 7.37-7.57, 9.47-9.51 and 11.47-11.57, particularly §§ 9.50-9.51, 11.12-11.13, 11.45-11.47 and 11.55-11.57).

As used herein, the term “moiety” refers to a specific group of atoms within a molecule, where the group of atoms is responsible for certain characteristic properties of that molecule. In the context of the present disclosure, a fluorescent moiety (e.g., a “fluorophore”) absorbs energy at a first wavelength and emits at a second, longer wavelength. A quencher moiety (e.g., a “quencher”) may have an absorption spectrum that overlaps the emission spectrum of a paired fluorophore. Quencher moieties typically release energy without emitting a photon. When a quencher moiety is close to a fluorophore (e.g., typically within 1-100 nm) that is emitting energy, the quencher can absorb and dissipate some or most of the emitted energy.

A “non-extendable” oligomer includes a blocking moiety at or near its 3′-terminus to prevent extension. A blocking group near 3′-end in some embodiments is within five residues of 3′-end and is sufficiently large to limit binding of a polymerase to the oligomer, and other embodiments contain a blocking group covalently attached to the 3′ terminus. Many different chemical groups can be used to block 3′-end, including alkyl groups, non-nucleotide linkers, alkane-diol dideoxynucleotide residues (e.g., 3′-hexanediol residues), and cordycepin. Further examples of blocking moieties include a 3′-deoxy nucleotide (e.g., a 2′,3′-dideoxy nucleotide); a 3′-phosphorylated nucleotide; a fluorophore, quencher, or other label that interferes with extension; an inverted nucleotide (e.g., linked to the preceding nucleotide through a 3′-to-3′ phosphodiester, optionally with an exposed 5′-OH or phosphate); or a protein or peptide joined to the oligonucleotide so as to prevent further extension of a nascent nucleic acid chain by a polymerase. A non-extendable oligonucleotide of the present disclosure can be at least 10 bases in length, and can be up to 15, 20, 25, 30, 35, 40, 50 or more nucleotides in length. Non-extendable oligonucleotides that comprise a detectable label can be used as probes.

A “template termination moiety” refers to a moiety in a template strand that prevents extension of an opposite-strand primer past the template termination moiety on the template strand. In some embodiments, a template termination moiety is included in the dual-specificity primer within or upstream of the joining region. After the dual-specificity primer is extended to form a first extension product, the first extension product serves as a template for extension of an opposite-strand primer. The template termination moiety prevents extension of the opposite-strand past the joining region, such that the formed amplicon resulting from extension of the opposite-strand does not include the complement of 5′-end insert-binding sequence of the dual-specificity primer.

As used herein, a biological “strain” is a sub-type of a genetic variant of a biological species. For example, a species of bacteria may occur and wildtype and drug-resistant strains. Methicillin-resistant Staphylococcus aureus (MRSA) and methicillin-sensitive Staphylococcus aureus (MSSA) are two strains of the species, Staphylococcus aureus.

An “insertion sequence” is a sequence found in the nucleic acid sequence of certain members of a species of organism, but not in other members of the species of organism. In some embodiments, the insertion sequence is a sequence that confers resistance to treatment with a particular therapy (e.g., an antibiotic). In some embodiments, the insertion sequence confers drug-resistance on Staphyloccus aureus. In some embodiments, the insertion sequence encodes a β-lactam-resistant penicillin-binding protein that confers resistance to β-lactam antibiotics including methicillin and penicillin. In some embodiments, the insertion sequence is a sequence juxtaposed to another sequence, which does not occur in all members of a species of organism. In some embodiments, the presence of the insertion sequence is indicative of a disease state. In some embodiments, the disease state is cancer.

A “chromosomal rearrangement” is a type of chromosome abnormality involving a change in the structure of the native (sometimes “wild-type”) chromosome. Such changes may involve deletions, duplications, inversions, and translocations. Usually, these events are caused by a breakage in the DNA double helices at two different locations, followed by a rejoining of the broken ends. This new juxtaposition of chromosomal sequences creates a “breakpoint” or “junction” between two sequences that are not ordinarily joined. Some chromosomal rearrangements are indicative of cancer in humans or laboratory animals, or drug resistance in microorganisms.

A “junction” is a point where two structures are joined together. For example, a junction can be the point where two sequences in a strand of nucleic acid are joined together or “juxtaposed.”

“MRSA nucleic acid” generally refers to a nucleic acid found in MRSA, including but not limited to orfX/SCCmec junction, mecA or mecC, and sequences indicative of S. aureus (e.g., S. aureus-specific sequences). “MRSA target nucleic acid” is a MRSA nucleic acid that is targeted for amplification and/or detection in a method according to this disclosure. “MRSA amplicon” refers to an amplicon produced from the amplification of MRSA nucleic acid.

An “orfX/SCCmec junction” comprises sequences (i.e., one or more nucleotides) from the S. aureus orfX gene joined directly to SCCmec sequence (i.e., one or more nucleotides), such as is formed by integration of SCCmec into the S. aureus chromosome. In some embodiments, an orfX/SCCmec junction comprises at least about 20 nucleotides of orfX sequence and at least about 20 nucleotides of SCCmec sequence. In some embodiments, an orfX/SCCmec junction comprises at least about 30, 40, 50, 60, 70, 80, 90, or 100 nucleotides of SCCmec sequence.

An “MREJ” (mec right extremity junction) is the SCCmec-derived portion of an orfX/SCCmec junction.

References, particularly in the claims, to “the sequence of SEQ ID NO: X” refer to the base sequence of the corresponding sequence listing entry and do not require identity of the backbone (e.g., RNA, 2′-O-Me RNA, LNA, or DNA) or base modifications (e.g., methylation of cytosine residues) unless otherwise indicated. Furthermore, T residues are understood to be interchangeable with U residues, and vice versa, unless otherwise indicated.

Unless otherwise indicated, “sense,” “positive-sense,” or “positive-strand” nucleic acid generally refers to the coding strand of an ORF (open reading frame) or non-coding nucleic acid on the same strand as the coding strand of the transcript, operon, mRNA, etc., of which it is a part, or otherwise of the closest ORF, and “antisense,” “negative-sense,” “negative-strand” nucleic acid refers to the complement of a “sense,” “positive-sense,” or “positive-strand” nucleic acid. Unless otherwise indicated, “hybridizing to a MRSA (or S. aureus) nucleic acid” includes hybridizing to either a sense or antisense strand thereof (e.g., either strand of a dsDNA MRSA sequence). Similarly, expressions such as “hybridization to a site comprising position X of SEQ ID NO: Y” and “competing for hybridization to SEQ ID NO: Y” can generally include hybridizing to either a sense or antisense strand of SEQ ID NO: Y; where a hybridized oligomer is configured to produce an amplicon, the proper orientation will be immediately apparent to one skilled in the art. The term “opposite-sense” simply refers to the sense or polarity of a second hybridized nucleic acid strand, without invoking limitations on the protein coding strand. An alternative approach for distinguishing strands of nucleic acid is to refer to “first” and “second” strands, also without invoking any protein coding functionality. An “opposite-strand” primer or oligonucleotide is one complementary or hybridizable to a reference nucleic acid strand (e.g., a first strand).

As used herein, the term “invasive cleavage structure” (or simply “cleavage structure”) refers to a structure comprising: (1) a target nucleic acid, (2) an upstream nucleic acid (e.g., an invasive probe oligonucleotide), and (3) a downstream nucleic acid (e.g., a primary probe oligonucleotide), where the upstream and downstream nucleic acids anneal to contiguous regions of the target nucleic acid, and where an overlap forms between a 3′ portion of the upstream nucleic acid and duplex formed between the downstream nucleic acid and the target nucleic acid. An overlap occurs where one or more bases from the upstream and downstream nucleic acids occupy the same position with respect to a target nucleic acid base, whether or not the overlapping base(s) of the upstream nucleic acid are complementary with the target nucleic acid, and whether or not those bases are natural bases or non-natural bases. In some embodiments, 3′ portion of the upstream nucleic acid that overlaps with the downstream duplex is a non-base chemical moiety such as an aromatic ring structure, as disclosed, for example, in U.S. Pat. No. 6,090,543. In some embodiments, one or more of the nucleic acids may be attached to each other, for example through a covalent linkage such as nucleic acid stem-loop, or through a non-nucleic acid chemical linkage (e.g., a multi-carbon chain). An invasive cleavage structure also is created when a cleaved 5′ flap hybridizes to a FRET cassette (i.e., when the “target nucleic acid” and the “downstream nucleic acid” are covalently linked in a stem-loop configuration). The “target nucleic acid” sequence of a FRET cassette that hybridizes to a cleaved 5′ flap can be referred to as a “5′ flap-hybridizing sequence.”

As used herein, an “INVADER® assay” or “invasive cleavage assay” refers to an assay for detecting target nucleic acid sequences in which an invasive cleavage structure is formed and cleaved in the presence of the target sequence. In some embodiments, reagents for an invasive cleavage assay include: a cleavage agent; and oligonucleotides (e.g., an “invasive probe,” a “primary probe,” and a “FRET cassette”). In some embodiments the invasive probe is an amplification oligomer or extension product thereof. The invasive cleavage assay can combine two invasive signal amplification reactions (i.e., a “primary reaction” and a “secondary reaction”) in series in a single reaction mixture. In some embodiments, detecting the presence of an invasive cleavage structure is achieved using a cleavage agent. The primary probe can be part of a probe system. In some embodiments, an additional portion of the primary probe comprises or consists of a 3′ terminal nucleotide which is not complementary to the target nucleic acid and/or which is non-extendable. In some embodiments, the additional portion of the primary probe is configured to interact with a FRET cassette to promote a cleavage reaction catalyzed by a flap endonuclease. In some embodiments, the reagents for an INVADER® assay further include a nuclease, such as a cleavase or a FEN enzyme (e.g., Afu, Ave, RAD2 or XPG proteins), or other enzyme (e.g., a DNA polymerase with 5′ nuclease activity, optionally with inactivated or reduced synthetic activity) where the nuclease has activity specific for a structure formed when both the invasive and primary probes are hybridized to a target sequence (e.g., a structure that can result when a duplex of the primary probe and the target undergoes 3′-end invasion by the invasive probe, where at least 3′ end and/or an intermediate portion of the invasive probe is hybridized, 5′ end of the primary probe is free, and an intermediate and/or 3′-terminal portion of the primary probe is hybridized). In some embodiments, the reagents for an INVADER® assay further comprise a buffer solution. In some embodiments, the buffer solution comprises a source of divalent cations (e.g., Mn²⁺ and/or Mg²⁺ ions, such as a magnesium salt or manganese salt. Example salts include MgCl₂, MnCl₂, magnesium acetate, manganese acetate, etc.). In some embodiments, the reagents for an INVADER® assay further comprise at least a third oligomer, such as at least one amplification oligomer that together with the first oligomer is configured to produce an amplicon (e.g., via PCR). In such embodiments, the primary probe can comprise a target-hybridizing sequence configured to specifically hybridize to the amplicon. In some embodiments, the reagents for an INVADER® assay further comprise amplification reagents, such as PCR reagents. Embodiments of an INVADER® assay in which the target sequence is amplified and monitored as the amplification reaction is occurring can be referred to as INVADER PLUS® assays. Including amplification in the assay can provide a lower limit of detection. INVADER® assays, cleavases, other nucleases, other possible INVADER®/INVADER PLUS® reagents, etc., are discussed, for example, in U.S. Pat. Nos. 5,846,717, 5,985,557, 5,994,069, 6,001,567, 6,913,881, 6,090,543, 7,482,127, and 9,096,893; International Pub. Nos. WO 97/27214, WO 98/42873, and WO 2016/179093; Lyamichev et al., Nat. Biotech., 17:292 (1999); and Hall et al., PNAS, USA, 97:8272 (2000).

As used herein, the term “thermostable” when used with reference to an enzyme, indicates that the enzyme is functional or active (i.e., can perform catalysis) at an elevated temperature (e.g., at about 55° C. or higher). In some embodiments, the enzyme is functional or active at an elevated temperature of 65° C. or higher (e.g., 75° C., 85° C., or even 95° C.). An exemplary “thermostable polymerase” can synthesize a new DNA strand in a template-dependent fashion at 75-80° C.

As used herein, the term “flap endonuclease” or “FEN” (e.g., “FEN enzyme”) refers to a class of nucleolytic enzymes that act as structure-specific endonucleases on DNA structures with a duplex containing a single-stranded 5′ overhang, or flap, on one of the strands that is displaced by another strand of nucleic acid, such that there are overlapping nucleotides at the junction between the single and double-stranded DNA. FEN enzymes catalyze hydrolytic cleavage of the phosphodiester bond 3′ adjacent to the junction of single and double stranded DNA, releasing the overhang, or “flap” (see Trends Biochem. Sci. 23:331-336 (1998) and Ann. Rev. Biochem. 73:589-615 (2004)). FEN enzymes may be individual enzymes, multi-subunit enzymes, or may exist as an activity of another enzyme or protein complex, such as a DNA polymerase. A flap endonuclease may be thermostable. Examples of FEN enzymes useful in the methods disclosed herein are described in U.S. Pat. Nos. 5,614,402, 5,795,763, and 6,090,606; and International Pub. Nos. WO 98/23774, WO 01/90337, WO 02/070755, and WO 03/073067. Particular examples of commercially available FEN enzymes include the CLEAVASE® enzymes (Hologic, Inc.).

“Cassette,” when used in reference to an INVADER® assay and/or invasive cleavage assay or reaction, as used herein refers to an oligomer or combination of oligomers configured to generate a detectable signal in response to cleavage of a detection oligomer in an INVADER® assay. In some embodiments, the cassette hybridizes to an cleavage product (e.g., a “flap”) from cleavage of the detection oligomer (e.g., primary probe). In some embodiments, such hybridization results in a detectable change in fluorescence. In some embodiments, such hybridization forms a second invasive cleavage structure, such that the cassette can then be cleaved. In some embodiments, a cassette comprises an interacting pair of labels, e.g., a FRET pair (in which case the cassette is a “FRET cassette”). In some embodiments, a FRET cassette undergoes a detectable change in fluorescence properties upon hybridization to the product from cleavage of the detection oligomer. For example, a FRET cassette can increase fluorescence emission at a first wavelength and/or decrease fluorescence emission at a second wavelength based on a change in the distance between labels upon hybridization to a cleavage product following cleavage of the detection oligomer. This can result from a decrease in energy transfer from a donor fluorophore (e.g., a decrease in quenching of a fluorophore, or a decrease in energy transfer from a donor fluorophore to an acceptor fluorophore). In some embodiments, a FRET cassette adopts a hairpin conformation, where the interaction of the pair of labels substantially suppresses (e.g., quenches) a detectable energy emission (e.g., a fluorescent emission). In some embodiments, a FRET cassette comprises a portion that hybridizes to a complementary cleaved 5′ flap of a primary probe to form an invasive cleavage structure that is a substrate for a cleavage agent (e.g., FEN enzyme). In some embodiments, cleavage of the FRET cassette by a cleavage agent separates the donor and acceptor moieties with the result of relieving the suppression and permitting generation of a signal.

As used herein, “S. aureus-specific sequence” refers to a sequence that distinguishes S. aureus from other staphylococci, including CNS. Non-limiting examples of S. aureus-specific sequences include sequences in the nuc, rRNA, femB, Sa442, Staphyloxanthin, GAPDH genes, and the like, of S. aureus which generally contain distinguishably different sequence in other Staphylococcus species. Discussion of S. aureus-specific sequence detection can be found, e.g., in Grisold et al., Methods Mol. Biol. 345:79-89 (2005); Costa et al., Diag. Microbiol. and Infect. Dis, 51:13-17 (2005); Mc Donald et al., J. Clin. Microbiol., 43:6147-6149 (2005); Zhang et al. J. Clin. Microbiol. 43:5026-5033 (2005); Hagen et al., Int J Med Microbiol. 295:77-86 (2005); Maes et al. J. Clin. Microbiol. 40:1514-1517 (2002); Saito et al., J. Clin. Microbiol. 33:2498-2500 (1995); Ubukata et al., J. Clin. Microbiol. 30:1728-1733 (1992); Murakami et al., J. Clin. Microbiol. 29:2240-2244 (1991); and Hiramatsu et al., Microbiol. Immunol. 36:445-453 (1992)).

An “S. aureus-specific amplicon” is an amplicon produced from an S. aureus-specific sequence. In general, production of an S. aureus-specific amplicon indicates the presence of S. aureus nucleic acid.

As used herein, a “kit” is a packaged combination of reagents, including one or more dual-specificity primers, as disclosed herein. For example, a kit can include a packaged combination of one or more vials, tubes, or cartridges having a plurality of chambers containing reagents for amplifying and detecting nucleic acids. The reagents can include oligonucleotide primers and probes such as those described herein, as well as nucleotide polymerizing enzymes (e.g., a DNA polymerase, a reverse transcriptase, an RNA polymerase, etc.). Optionally, a flap endonuclease (FEN) enzyme also can be included in the kit. In certain preferred embodiments, the reagents can be in liquid form, in solid form (e.g., a lyophilisate), or a semi-solid form (e.g., a glass). In some embodiments, oligonucleotide reagents and enzyme reagents are present in the kit as components of a single lyophilized composition (e.g., a pellet). In such an instance, primers, probes, and one or more enzymes (e.g., a DNA polymerase and/or a FEN enzyme) can be disposed in the same reaction chamber or vessel in a lyophilized form that can be reconstituted with an aqueous reagent, where a separate vial or tube containing the aqueous reagent is included in the same kit. The kits may further include a number of optional components such as, for example, capture probes (e.g., poly-(k) capture probes as described in US 2013/0209992). Other reagents that may be present in the kits include reagents suitable for performing in vitro amplification such as buffers, salt solutions, and/or appropriate nucleotide triphosphates (e.g., dATP, dCTP, dGTP, dTTP; and/or ATP, CTP, GTP and UTP). Kits further can include a solid support material (e.g., magnetically attractable particles, e.g., magnetic beads) for immobilizing the capture probe, either directly or indirectly, in a sample-preparation procedure. In certain embodiments, the kit further includes a set of instructions for practicing methods in accordance with the present disclosure, where the instructions may be associated with a package insert and/or the packaging of the kit or the components thereof.

References herein to “first” and “second” and “third” etc., (e.g., oligonucleotides, amplicons, etc.) simply provide identifiers for distinguishing one from another, without necessarily indicating one precedes the other.

Unless defined otherwise, all scientific and technical terms used herein have the same meaning as commonly understood by those skilled in the relevant art. General definitions can be found in technical books relevant to the art of molecular biology, e.g., DICTIONARY OF MICROBIOLOGY AND MOLECULAR BIOLOGY, 2nd ed. (Singleton et al., 1994, John Wiley & Sons, New York, NY) or THE HARPER COLLINS DICTIONARY OF BIOLOGY (Hale & Marham, 1991, Harper Perennial, New York, NY).

### Introduction and Overview

Disclosed herein are techniques employing structured primers referred to as “dual-specificity primers.” These primers permit amplification of nucleic acid sequences only when the template includes two sequences (e.g., juxtaposed or joined sequences) that are both hybridized by the dual-specificity primer, where exponential amplification can occur on only one side of a junction between the two sequences. This is because the portion of the dual-specificity primer hybridizing downstream of the junction between the two sequences is precluded from serving as a template for extension of any opposite-strand primer due to the presence of a template termination moiety. The dual-specificity primer is configured such that it includes a joining region that links two regions of the primer that hybridize to the template nucleic acid, where the joining region does not stably hybridize to the sequence of the template nucleic acid during initial polymerase-based extension of the primer. In some preferred embodiments, the joining region does not stably hybridize to the sequence of any nucleic acid in an amplification reaction mixture during initial polymerase-based extension of the primer. A template termination moiety positioned within or upstream of the joining region (e.g., where the upstream portion of the dual-specificity primer and the joining region meet) functions as a stop for polymerase-based extension of opposite-strand primers. Advantageously, dual-specificity primers can amplify nucleic acid sequences even when there is variability in the spacing between the two sequences that are hybridized by the dual-specificity primer (i.e., by the 5′ sequence of nucleotides and 3′ sequence of nucleotides positioned on opposite sides of the joining region). Thus, there can be variability in the location of the junction in the target nucleic acid (i.e., the template for primer extension). Preferably, dual-specificity primers are not used to detect the presence or absence of single nucleotide differences in the region of a template nucleic acid that is hybridized by 3′ sequence of nucleotides of the dual-specificity primer.

Template nucleic acids used to illustrate the disclosed technique herein included a junction to be detected, but the origin of the junction sequence (e.g., how the junction was created) was unimportant for successful use of the dual-specificity primer. Generally speaking, a “junction” between two sequences can be recognized as a feature where two different nucleic acids (e.g., “first” and “second” nucleic acids) differ from each other insofar as sharing a common sequence (or substantially similar sequence) adjacent to sequences that substantially differ between the two nucleic acids. Nucleic acid junctions may result from alternative RNA splicing or polyadenylation events that select or join exon sequences during post-transcriptional processing. Alternatively, junctions may occur in genomic nucleic acids (e.g., procaryotic or eucaryotic DNA, viral RNA, etc.), and may reflect gene rearrangements or integration or insertion events.

Detection of juxtaposed sequences is illustrated herein using a model system where bacterial genomic DNA either included or did not include a nucleic acid junction. More particularly, the model system involved detection of a junction between model bacterial chromosomal target sequences and a mobile insert that encodes resistance to an antibiotic (i.e., methicillin). The mobile insert is known to integrate into a specific gene of the S. aureus chromosome, although at a somewhat divergent position. Some strains of the species include the insert sequence (e.g., MRSA), while other strains of the species (e.g., MSSA) do not. Ready availability of both closely related organisms provided a convenient way to demonstrate utility of the dual-specificity primer.

Primer sets that included a dual-specificity primer were used herein in a method of selectively amplifying and detecting a target nucleic acid of a first bacterial strain (i.e., MRSA). This target nucleic acid (sometimes the “first nucleic acid”) of the first bacterial strain included an insertion sequence not found in the nucleic acid (sometimes the “second nucleic acid”) of a second bacterial strain (i.e., MSSA). The ordinary skilled artisan will appreciate that an insertion sequence harboring the mecA gene or the mecC gene is inserted into the orfX gene of the S. aureus bacterial chromosome to confer resistance to methicillin. Accordingly, a junction between the insertion sequence and the orfX chromosomal sequence distinguishes the two bacterial strains (MRSA and MSSA) from each other. Nucleic acids from both strains harbor orfX sequences (e.g., “common sequences”), but only the MRSA strain harbors the insertion sequence. For simple reference, distinct sequences (representing the insertion sequence or the wild-type S. aureus chromosomal sequence) were positioned downstream of the common sequence shared by the first (MRSA) and second (MSSA) nucleic acids. The dual-specificity primer included: (i) a 5′ sequence of nucleotides that stably hybridized to a first sequence (e.g., an insertion sequence) present in the nucleic acid of MRSA but not the nucleic acid of MSSA; (ii) a 3′ sequence of nucleotides complementary to a sequence (i.e., the “common sequence”) substantially similar in the nucleic acids of both the first and second bacterial strains; and (iii) a joining region that joined 3′ sequence of nucleotides to 5′ sequence of nucleotides. The joining region of the dual-specificity primer did not stably hybridize to any nucleic acid sequence that seperated the common sequence (sometimes “common first sequence”) and second downstream nucleic acid sequences contained in nucleic acids of the two bacterial strains under the amplification conditions.

In some preferred embodiments, the joining region includes at least one non-nucleotide moiety that functions as a template-termination moiety. For example, dual-specificity primers illustrated herein included a non-nucleotide linker or spacer at the junction of 5′ sequence and the joining region. Positioning of the template termination moiety within the joining region can be varied to adjust the Tm profile of the primer during an exponential amplification reaction subsequent to a first complete cycle. The method further included detecting the formation of amplicon in the sample under the amplification conditions as an indication that the first nucleic acid (e.g., the first bacterial strain) was present in the sample or, alternatively, detecting the absence of formation of amplicon in the sample under the amplification conditions as an indication that the first nucleic acid was not present in the sample.

In the context of the present disclosure, and with reference to the common sequences present in the different nucleic acid targets (e.g., represented by MRSA and MSSA), “substantially similar” means at least 80% identical over the length of the compared sequences. Likewise, “substantially complementary” means at least 80% complementary bases over the length of the compared sequences. Practically speaking, there can be up to a single nucleotide difference between two sequences up to 9 nucleotides in length, and up to 2 nucleotide differences between two sequences from 10 to 14 nucleotides in length. Preferably, 3′ sequence of nucleotides of the dual-specificity primer is fully complementary to the common sequences found in the nucleic acids of the first and second bacterial strains. In the illustrative Examples, 3′ sequence of nucleotides of the dual-specificity primer was substantially complementary to the orfX sequences found in both MRSA and MSSA.

In accordance with the disclosure, 3′ sequence of nucleotides of the dual-specificity primer stably hybridized to the common sequence of the first nucleic acid (e.g., nucleic acid of MRSA) only when 5′ sequence of nucleotides of the dual-specificity primer (e.g., the insert-binding sequence) was stably hybridized to the same nucleic acid strand. Thus, it was possible to selectively amplify the target nucleic acid from MRSA because 3′ sequence of nucleotides of the dual-specificity primer did not stably hybridize to the common sequence of the second nucleic acid (e.g., nucleic acid of MSSA). Again, in the absence of stable hybridization of 5′ sequence of nucleotides of the dual-specificity primer, it is believed that there could be no appreciable hybridization of 3′ sequence of nucleotides of the dual-specificity primer that was required for a polymerase-based amplification reaction (e.g., PCR).

In some illustrative embodiments, the insertion sequence present in one of two bacterial strains encodes a protein that confers drug-resistance on the organism. In some embodiments, the insertion sequence is a β-lactam-resistant penicillin-binding protein that confers resistance to β-lactam antibiotics. In some embodiments, the insertion sequence includes a mecA or mecC gene. In some embodiments, the insertion sequence includes an SCCmec right extremity junction (MREJ) sequence. In some such embodiments, the MREJ is a type i, ii, iii, iv, v, vi, vii, viii, ix, xii, xiii, iv, xv, or xxi SCCmec MREJ sequence. When present, an additional oligomer (e.g., a detection oligomer) can be configured to specifically hybridize to an amplicon produced by the primer set.

In some illustrative embodiments detailed below, 3′-end target-binding sequence of the dual-specificity primer is complementary to a sequence contained in S. aureus nucleic acid. In some embodiments, the sequence contained in S. aureus nucleic acid comprises an orfX sequence. The orfX sequence is a nucleic acid sequence common to MRSA and MSSA.

In some embodiments, a nucleic acid sequence contained in the target nucleic acid of first members of a species of organisms encodes a fusion protein that is indicative of a disease state and the insertion sequence encodes a portion of the fusion protein. In some embodiments, the target nucleic acid sequence contained in the target nucleic acid of first members of the species of organisms comprises a nucleic acid rearrangement that is indicative of a disease state, and the insertion sequence encodes one member of the nucleic acid rearrangement. In certain embodiments, the disease state is a type of cancer. In certain embodiments, the indicative nucleic acid rearrangement can be the BCR-ABL fusion.

The BCR-ABL fusion oncogene results when segments of human chromosomes 9 and 22 exchange places to form the “Philadelphia chromosome.” This translocation is found in most patients with chronic myelogenous leukemia (CML), and in some patients with acute lymphoblastic leukemia (ALL) or acute myelogenous leukemia (AML). Dual-specificity primers can be used to amplify nucleic acids characteristic of the juxtaposed BCR and ABL sequences in the Philadelphia chromosome. More specifically, a 5′ insert-binding sequence of a dual-specificity primer can be complementary to a sequence on one side of a BCR-ABL junction, and a 3′ target-binding sequence of the primer can be complementary to a sequence on the other side of the BCR-ABL junction, with the joining region of the dual-specificity primer spanning the junction between BCR and ABL sequences. The BCR-ABL fusion protein encoded by the fusion oncogene is known to exhibit enhanced tyrosine kinase activity that is believed to be responsible for the tumor phenotype.

In other embodiments, dual-specificity primers in accordance with the disclosure are used for amplifying nucleic acids upstream of a sequence that includes a junction between two sequences, where the two sequences are joined or juxtaposed in cells of a tumor. Again, the Philadelphia chromosome represents such an example, where the BRC and ABL gene sequences that are ordinarily present on different chromosomes are juxtaposed as the result of a translocation. The tumor can be a malignant tumor (i.e., cancer). The two sequences may be brought together in the genome as a consequence of a gene rearrangement.

In other embodiments, dual-specificity primers in accordance with the disclosure can be used to amplify nucleic acids upstream of a junction between exon sequences that are spliced together during post-transcriptional RNA processing. In this instance, 5′ sequence of the dual-specificity primer can hybridize to a sequence located in a first exon having a sequence that distinguishes RNA splicing variants from each other. The 3′ sequence of the dual-specificity primer can hybridize to a sequence located in a different exon that is brought into juxtaposition with the first exon as the result of RNA splicing. In some preferred embodiments, the 3′ sequence of the dual-specificity primer hybridizes to a common exon sequence that is shared between the RNA splicing variants.

### Exemplary Compositions, Kits, Methods, and Uses

The present disclosure provides oligomers (e.g., dual-specificity primers), compositions, and kits, useful for amplifying and detecting target nucleic acids from a sample. Kits typically include, in one or more vials or containers, at least one dual-specificity primer, and optionally an opposite-strand primer that hybridizes to an extension product of the at least one dual-specificity primer when extended by a polymerase on a template nucleic acid strand of a target nucleic acid.

Dual-specificity primers in accordance with the disclosure include two target-hybridizing sequences separated from each other by a joining region that includes a template termination moiety. A first target-hybridizing sequence (i.e., the “5′ sequence” or “5′ insert-binding sequence”) is positioned upstream of the template termination moiety and joining region, which are positioned upstream of a second target-hybridizing sequence (i.e., the “3′ sequence” or “3′ target-binding sequence”). The second target-hybridizing sequence terminates at its 3′-end with a hydroxyl group that can be extended by a polymerase in a template-dependent fashion. Preferably, the template termination moiety is positioned downstream and adjacent to the first target-hybridizing sequence. The joining region is upstream of, and adjacent to the second target-hybridizing sequence. In some embodiments, the joining region includes nucleotides or non-nucleotide moieties that do not hybridize the template strand of the target nucleic acid by complementary base pairing. In some embodiments, the template termination moiety is a non-nucleotide moiety. One example of a template termination moiety is an internal “C9” linker or “spacer” that comprises a glycol linkage. In other embodiments, the template termination moiety comprises a nucleotide in an inverted orientation that precludes reading by a polymerase, and so functions as a polymerase termination point. Optionally, this inverted orientation is achieved using either or both of a 3′-3′ or a 5′-5′ linker in the oligonucleotide structure.

Generally speaking, the Tm characterizing the binding or hybridization of the upstream or 5′ sequence of a dual-specificity primer to the template strand of the target nucleic acid is higher than the Tm characterizing the binding or hybridization of 3′ sequence to the template strand. This means that 3′ sequence of the dual-specificity primer will dissociate from the template strand of the target nucleic acid at a temperature lower than the temperature at which 5′ sequence of the dual-specificity primer will dissociate from the template strand. The 5′ sequence of a dual-specificity primer preferably is longer than 3′ sequence of the dual-specificity primer. Subject to these Tm and length constraints, the length of 5′ sequence of the dual-specificity primer preferably falls in the range of from 10-60 nts, and the 3′ sequence of the dual-specificity primer preferably falls in the length range of from 6-10 nts. More preferably, the length of 5′ sequence of the dual-specificity primer falls in the range of from 10-45 nts, more preferably in the range of from 11-30 nts, and still more preferably in the range of from 11-25 nts.

When used in an amplification reaction to detect two spaced-apart sequences in a nucleic acid strand, the dual-specificity primer can be configured such that the joining region spans across a junction or stretch of nucleotides between the two sequences. Preferably, the joining region of the dual-specificity primer does not interact with the template strand of the target nucleic acid to form a hybrid duplex structure by complementary base pairing. In some embodiments, the joining region of the dual-specificity primer includes a scrambled or randomized nucleotide sequence. In some embodiments, the joining region of the dual-specificity primer has a length in the range of from 5-50 nts, 5-25 nts, or 5-10 nts. Exemplary dual-specificity primers described herein had joining regions of 9 nts in length, but the primers could be hybridized to template nucleic acid strands with looped-out sequences (i.e., unhybridized sequences) of different lengths. In some embodiments, the joining region is of a length different from the length of nucleotides separating the template sequences complementary to the first- and second-target hybridizing sequences, as illustrated below in Example 2. In all instances 5′ sequence and 3′ sequence of a dual-specificity primer hybridized to the template strand on opposite sides of a junction.

In use, the dual-specificity primer is configured for stable hybridization to form a stable duplex that can be extended by a polymerase (e.g., a DNA polymerase) only when both of the target-hybridizing sequences on either side of the joining region are hybridized to the template strand. Although it is preferred, there is no requirement for full sequence complementarity between 5′ and 3′ sequences of nucleotides of the dual-specificity primer and the corresponding hybridized sequences in the template strand to form the stable duplex. As a consequence, the shorter second target-hybridizing sequence optionally may form a hybrid when there is perfect complementarity, or alternatively when there is up to one, or even up to two nucleotide mismatches between the sequence of the template strand of the target nucleic acid and 3′ sequence of the dual-specificity primer. For example, in some embodiments the 3′ sequence of the dual-specificity primer is 6-10 nts in length, and there can be up to two base mismatches that will be tolerated and still permit extension of the dual-specificity primer by a polymerase in a template-dependent fashion when the first target-hybridizing sequence also is hybridized to the same target nucleic acid strand. In some embodiments, a single base mismatch between the template strand and 3′ target-binding sequence of the dual-specificity primer will not prevent extension of the dual-specificity primer by a polymerase in a template-dependent fashion. In some embodiments, one base mismatch, but not two base mismatches between the template strand of the target nucleic acid and 3′ target-binding sequence of the dual-specificity primer will not prevent extension of the dual-specificity primer by a polymerase in a template-dependent fashion. In some instances, preferred dual-specificity primers can participate in amplification reactions when there is imperfect complementarity between 3′ sequence and the template strand, but again, only when 5′ sequence of the primer also is hybridized to the same target nucleic acid strand. In still other embodiments, there must be 100% complementarity between the base sequences of the target nucleic acid and 3′ sequence of the dual-specificity primer. In order for 3′ sequence of a dual-specificity primer to extend efficiently by the activity of a polymerase, it is important that the 3′ terminal base of the primer is complementary to the corresponding base in the template strand.

In some embodiments, a pair of oligomers is provided where one oligomer is configured to hybridize to a sense strand of a target nucleic acid and the other is configured to hybridize to an anti-sense or “opposite-sense” strand of the target nucleic acid. Preferrably, at least one of the oligos of the pair is a dual-specificity primer. Such oligomers include primer pairs for PCR or other forms of amplification. In some embodiments, one or more oligomers, such as a primer set (defined as at least two primers configured to generate an amplicon from a target sequence) or a primer set and an additional oligomer (e.g., detection oligomer) which is optionally non-extendible and/or labeled (e.g., for use as a primary probe or part of a probe system, optionally including a FRET cassette), are configured to hybridize to a target nucleic acid.

In some embodiments, the primer set is used in a method of detecting a target nucleic acid sequence contained in a target nucleic acid of first members of a species of organism suspected of being present in a sample, the target nucleic acid sequence including an insertion sequence not found in a nucleic acid sequence contained in a nucleic acid of second members of the species of organisms. A nucleic acid junction is created as a result of the insertion. The method comprises exposing or contacting the sample to the set of primers, where the set of primers includes at least one dual-specificity primer that comprises: (i) a 5′-end insert-binding sequence that stably binds to the insertion sequence of the target nucleic acid under the amplification conditions; (ii) a 3′-end target-binding sequence that is complementary to the target nucleic acid sequence contained in the target nucleic acid and in a nucleic acid sequence contained in the second members of the species of organism, where the target-binding sequence stably binds to the target nucleic acid sequence but not to the nucleic acid sequence contained in the second members of the species of organisms under the amplification conditions; and (iii) a joining region that joins 3′-end of the insert-binding sequence to 5′-end of the target-binding sequence, and where the joining region does not stably bind to any nucleic acid sequence contained in nucleic acids of the first or second members of the species of organisms under the amplification conditions, and the dual-specificity primer includes at least one non-nucleotide moiety upstream of the joining region. The method further comprises detecting the formation of amplicon in the sample under the amplification conditions as an indication that first members of the species of organisms are present in the sample or, alternatively, detecting the absence of formation of amplicon in the sample under the amplification conditions as an indication that first members of the species of organisms are not present in the sample. In some other embodiments, the method further comprises detecting amplicon formation after the amplification reaction is complete (i.e., so-called “end-point” detection).

In some embodiments, the insertion sequence encodes a protein that confers drug-resistance on the organism. In some embodiments, the insertion sequence is a β-lactam-resistant penicillin-binding protein that confers resistance to β-lactam antibiotics. In some embodiments, the insertion sequence includes a mecA or mecC gene. In some embodiments, the insertion sequence includes an SCCmec right extremity junction (MREJ) sequence. In some such embodiments, the MREJ is a type i, ii, iii, iv, v, vi, vii, viii, ix, xii, xiii, iv, xv, or xxi SCCmec MREJ sequence. When present, the additional oligomer (e.g., detection oligomer) can be configured to specifically hybridize to an amplicon produced by the primer set.

In some embodiments, 3′ sequence of the dual-specificity primer is complementary to a sequence contained in S. aureus nucleic acid. In some embodiments, the sequence contained in S. aureus nucleic acid includes an orfX sequence that is common to MRSA and MSSA.

The dual-specificity primer provides the specificity to indicate the presence of an insertion sequence in the nucleic acid of a particular species of an organism. The dual-specificity primer can distinguish between organisms of a particular species that contain the insertion sequence, and organisms of that species that do not contain the insertion sequence. The dual-specificity primer can also distinguish the target organism from different species of organisms that may also contain the insertion sequence.

Certain insertion sequences do not always integrate into the nucleic acid (e.g., chromosomal nucleic acid) of an organism at precisely the same location. As a result, there can be variation in the spacing between sequences characteristic of the organism and sequences characteristic of the insert. Generally speaking, the joining region, which spans the junction between a chromosomal target and an insertion sequence can accommodate such variation. For example, if the insertion site varies by 15 nucleotides, 5′-end insert-binding sequence of the dual-specificity primer can be configured to hybridize to an insertion nucleic acid sequence that is downstream of the 15-nucleotide insertion variation site in the target nucleic acid, and the 3′-end target-binding sequence of the dual-specificity primer can be configured to hybridize to a target nucleic acid sequence that is upstream of the 15 nucleotide variation site in the target nucleic acid. The 15-nucleotide variation site will not be involved in hybridization with the dual-specificity primer, because the joining region between 5′-end insert-binding sequence and 3′-end target-binding sequence does not hybridize to the target nucleic acid sequence. As evidenced by the results presented below in Example 2, there can be differences between the length of the joining region, and the length of the sequence separating the two binding sites for the dual-specificity primer in the target nucleic acid.

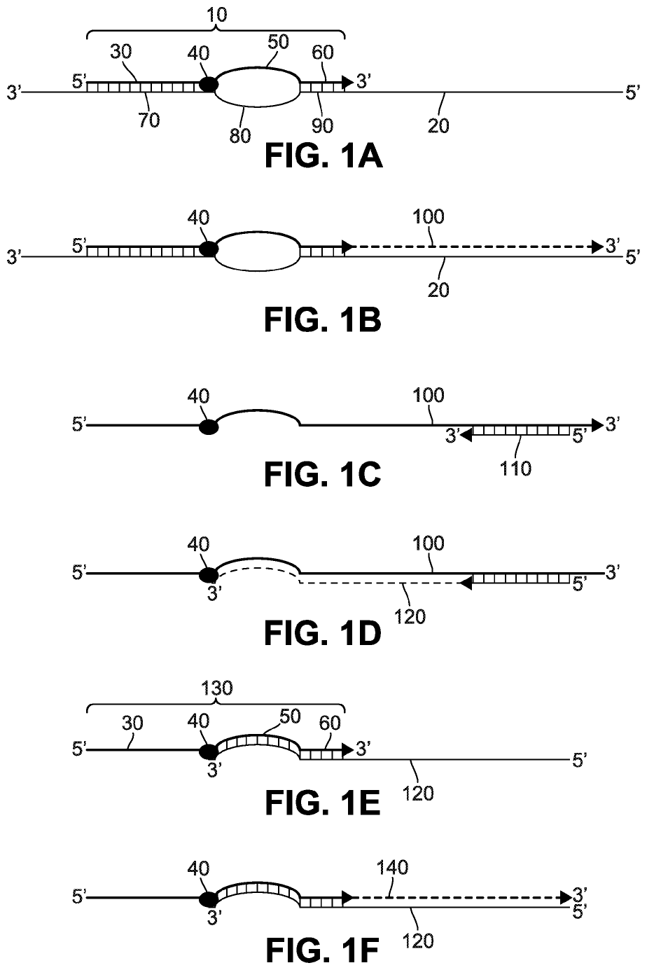

Methods of using dual-specificity primers in an amplification reaction (e.g., PCR) can be understood with reference to FIG. 1A-FIG. 1F. In these illustrative embodiments, a 5′ sequence of nucleotides 30 (e.g., an insert-binding sequence) of a dual-specificity primer 10 is longer than a 3′ sequence of nucleotides 60 (e.g., a target-binding sequence) of the dual-specificity primer 10, and 5′ sequence of nucleotides 30 is separated from 3′ sequence of nucleotides 60 by a “joining” region 50 that does not hybridize to the target nucleic acid 20. The 5′ sequence of nucleotides 30 of the dual-specificity primer 10 can stably hybridize to a downstream sequence 70 (e.g., an insertion sequence) that may be present in the target nucleic acid 20 under hybridization conditions. The 3′ sequence of nucleotides 60 of the dual-specificity primer 10 can stably hybridize to an upstream sequence 90 of the target nucleic acid 20 only when 5′ sequence of nucleotides 30 of the primer is also stably hybridized to the same nucleic acid strand. The illustrated dual-specificity primer 10 further includes a template termination moiety 40, shown by the filled circle at the junction between 5′ sequence of nucleotides 30 and the joining region 50. The joining region 50 remains substantially single-stranded when the dual-specificity primer 10 is hybridized to the target nucleic acid 20. An unhybridized portion 80 of target nucleic acid 20, which may be as small as a single nucleotide, is opposite the joining region 50 of the dual-specificity primer 10 in a complex that includes the target nucleic acid 20 and the dual-specificity primer 10 (FIG. 1A). When 3′ sequence of nucleotides 60 of the dual-specificity primer 10 stably hybridizes to an upstream sequence 90 of the target nucleic acid 20, a polymerase enzyme (e.g., a DNA polymerase or a reverse transcriptase) can extend the dual-specificity primer 10 at its 3′-end using the target nucleic acid sequence 20 as a template to produce a first dual-specificity primer extension product 100 that includes the full sequence of the dual-specificity primer 10 (FIG. 1B). Prior to strand separation, the first dual-specificity primer extension product 100 and the target nucleic acid 20 are part of a complex where the joining region 50 of the extended dual-specificity primer remains substantially single-stranded (FIG. 1B).

Following strand separation, an opposite-strand primer 110 can hybridize to the first dual-specificity primer extension product 100 by complementary base pairing (FIG. 1C). The opposite-strand primer 110 can be extended by the activity of a polymerase using the first dual-specificity primer extension product 100 as the template, thereby producing an opposite-strand primer extension product 120 (e.g., “amplicon”). Template-dependent extension of the opposite-strand primer 110 does not proceed beyond the template termination moiety 40 that separates 5′ sequence of nucleotides 30 and the joining region 50 in a sequence characteristic of the dual-specificity primer 10 (FIG. 1D). Consequently, 5′ sequence of nucleotides 30 of the dual-specificity primer 10 that is included in the sequence of the first dual-specificity primer extension product 100 does not serve as a template for extension of the opposite-strand primer, and so remains single-stranded (FIG. 1D). Following another round of strand separation, and following hybridization of a fresh dual-specificity primer 130 to the opposite-strand primer extension product 120, 5′ sequence of nucleotides 30 of the fresh dual-specificity primer 130 does not hybridize to any amplification product produced in the amplification reaction. In some preferred embodiments, the dual-specificity primer 10 shown in FIG. 1A can be identical to the fresh dual-specificity primer 130 shown in FIG. 1E. The 5′ sequence of nucleotides 30 of the fresh dual-specificity primer 130 remains single-stranded while the joining region 40 and 3′ sequence of nucleotides 60 of the fresh dual-specificity primer 130 can be in a double-stranded configuration when hybridized to the opposite-strand primer extension product 120 (FIG. 1E). This contrasts with the single-stranded configuration of the joining region 50 of dual-specificity primer 10 hybridized to the target nucleic acid 20 (FIG. 1A). Extension of the fresh dual-specificity primer 130 using the opposite-strand primer extension product 120 as a template results in a second amplicon 140, where the second amplicon 140 includes the sequence of the fresh dual-specificity primer 130 (FIG. 1F). In some embodiments, the single-stranded 5′ sequence of nucleotides in the second amplicon 140 can serve as a binding sequence for hybrid capture immobilization (e.g., by capture onto a bead or other solid support), or detection (e.g., by hybridization of a complementary “probe” sequence, optionally including a detectable label attached thereto). In some alternative embodiments (not pictured), the fresh primer that hybridizes to opposite-strand primer extension product 120 includes all or part of the sequence of joining region 50 and optionally 3′ sequence of nucleotides 60 of dual-specificity primer 10.

In preferred applications, 5′ sequence of nucleotides of the dual-specificity primer stably hybridizes to the target nucleic acid only when a complementary, or substantially complementary sequence is contained in a downstream portion of the target nucleic acid. When the 5′ sequence of nucleotides of the dual-specificity primer is stably hybridized to the target nucleic acid, the shorter 3′ sequence of nucleotides of the dual-specificity primer will stably hybridize to the target sequence, also if a complementary or substantially complementary sequence match is present. If the complement of 5′ sequence of nucleotides of the dual-specificity primer is not present in the target nucleic acid of the sample, neither 5′ sequence of nucleotides nor the shorter 3′ sequence of nucleotides of the dual-specificity primer will stably hybridize to the target nucleic acid in the sample, even if the complement of 3′ sequence of nucleotides of the dual-specificity primer is present in the target nucleic acid. Also, if 5′ sequence of nucleotides of the dual-specificity primer stably hybridizes to its complementary sequence in the target nucleic acid, but the complement of 3′ sequence of nucleotides of the dual-specificity primer is not present, 3′ sequence of nucleotides will not stably hybridize to the target nucleic acid, and there will be no primer extension. Thus, the dual-specificity primer provides specificity for amplifying a nucleic acid sequence characteristic of members of a species that include two complementary (e.g., upstream and downstream) sequences in a spaced-apart arrangement, where the two sequences can be separated in the target nucleic acid by at least one nucleotide.

In some preferred embodiments, dual-specificity primers in accordance with the disclosure can be used to preferentially amplify one of two different target nucleic acids (e.g., “first” and “second” nucleic acids). The two target nucleic acids can share a common first sequence (e.g., a substantially similar sequence) in their upstream regions, and have different sequences in their downstream regions. Referring to FIG. 1, 3′ sequence of nucleotides of the primer (illustrated diagrammatically by element 60) preferably is substantially complementary to the common sequence shared by the first and second nucleic acids. The shared sequence may be represented by the upstream sequence 90 illustrated in the target nucleic acid of the figure. The 5′ sequence of nucleotides of the primer (illustrated diagrammatically by element 30) can be sufficiently complementary to the downstream sequence of one of the two nucleic acids (illustrated diagrammatically by element 70) as to permit hybridization only to that nucleic acid (e.g., hybridization to the first nucleic acid and not to the second nucleic acid). Since hybridization and subsequent extension of 3′ sequence of nucleotides of the dual-specificity primer depends on hybridization of 5′ sequence of nucleotides of the dual-specificity primer, amplification can be used for detecting target nucleic acids having juxtaposed sequences, or nucleic acid junctions.

Those having an ordinary level of skill in the art of primer design will appreciate that the length and sequence of 3′ sequence of a dual-specificity primer can be modified to optimize functional characteristics of the primer. For example, a 3′ sequence in the length range of from 6-10 nts, 6-8 nts, 5-8 nts, or exactly 6 nts can be used to differentially amplify target nucleic acid sequences differing in the sequence complementary to 3′ sequence of the dual-specificity by a single nucleotide. The 5′ sequence of the dual-specificity primer will typically be longer than 3′ sequence of the primer, and will be characterized by a higher Tₘ. For example, 5′ sequence of the dual-specificity primer preferably falls in the length range of from 10-60 nts, more preferably from 11-45 nts, more preferably 11-30 nts, and still more preferably from 11-25 nts. For example, 5′ sequence of the dual-specificity primer can have a length of 11 nts, 14 nts, 19 nts, 21 nts, or 23 nts. In some embodiments, destabilization of a hybrid duplex that includes 3′ sequence of the dual-specificity primer, for example caused by the presence of a single non-complementary base, can be sufficient to prevent the dual-specificity primer from extending and amplifying a template nucleic acid sequence. Conversely, perfect complementarity between 3′ sequence of the dual-specificity primer and the sequence of the target nucleic acid can lead to efficient amplification of the target nucleic acid sequence if the primer is also hybridized to the target nucleic acid at 5′ sequence of the dual-specificity primer. In some embodiments, varying the length and sequence of 3′ sequence provides a simple and convenient way to optimize functional characteristics of the dual-specificity primer. Disclosed below is an example where a dual-specificity primer having a 6 base 3′ sequence advantageously amplified one target nucleic acid sequence with specificity, but did not amplify another target nucleic acid sequence with a single mismatch to the 3′ sequence of the dual-specificity primer. A dual-specificity primer having a 3′ sequence 9 bases long amplified both target sequences, and so exhibited reduced specificity, even though the 5′ sequence of the dual-specificity primer mismatched the sequence of the target nucleic acid. In certain preferred embodiments, there is full complementarity between 5′ sequence of the dual-specificity primer (i.e., the sequence upstream of the template termination moiety) and the corresponding target nucleic acid sequence.

While 5′ sequence of a dual-specificity primer stably hybridizes to a downstream sequence of the target nucleic acid, and 3′ sequence of the dual-specificity primer stably hybridizes to an upstream sequence of the target nucleic acid to produce an amplicon in an amplification reaction, the conditions for stable hybridization of these two regions can be different. In some embodiments, each of 5′ sequence and 3′ sequence will be characterized by different Tms, but the Tm of the hybrid between 3′ sequence and the complementary target nucleic acid serving as a template preferably will be lower than the Tm of the hybrid between 5′ sequence and the downstream sequence of the target nucleic acid serving as a template. The ordinary skilled artisan will understand that “Tm” refers to the temperature at which 50% of the complementary sequences are hybridized in a dynamic equlibrium. When the Tms are arranged in this fashion, 5′ sequence generally binds with higher stability compared with 3′ sequence. This can permit fine sequence selectivity based on the base composition of 3′ sequence. Thus, in accordance with some embodiments, the Tm of 5′ sequence interaction with the downstream sequence of the target nucleic acid is higher than the Tm of 3′ sequence interaction with the target nucleic acid. For example, in some embodiments, these two Tms may differ by any of at least about 1° C., at least about 2° C., about 5° C., about 1° C. to about 5° C., or about 3° C. to about 10° C.

Suitable template termination moieties for the dual-specificity primers will be known to those of skill in the art. In some embodiments, a suitable template termination moiety is a non-nucleotide spacer, preferably several atoms (e.g., at least 9 atoms) in length. In some embodiments, a suitable template termination moiety is commercially available as a hexethylene glycol or an abasic nucleotide that lacks a nitrogenous base. In some embodiments, a suitable template termination moiety is incorporated into the dual-specificity primer structure as a phosphoramidite reagent during oligonucleotide synthesis. Examples include variously sized “spacer” phosphoramidites (e.g., 9-O-Dimethoxytrityl-triethylene glycol, 1-[(2-cyanoethyl)-(N,N-diisopropyl)]-phosphoramidite (“C9 spacer” herein); 12-(4,4′-Dimethoxytrityloxy) dodecyl-1-[(2-cyanoethyl)-(N,N-diisopropyl)]-phosphoramidite; etc.) commercially available from Glen Research (Sterling, VA). In some embodiments, a suitable template termination moiety includes one or more nucleotides in an inverted orientation. A sequence of inverted nucleotides can be created, for example, by synthesizing the dual-specificity primer so that it includes 3′-3′ and 5′-5′ linkers flanking the “inverted” nucleotides.

In some embodiments, at least one opposite-strand primer of the primer set hybridizes to a S. aureus-specific target sequence. In an exemplary embodiment, the S. aureus-specific sequence is a sequence in the orfX gene. Alternatives include the nuc, rRNA, femB, Sa442, Staphyloxanthin, or GAPDH genes of S. aureus. In some embodiments, the S. aureus-specific sequence is a sequence in the GAPDH gene of S. aureus. In some embodiments, amplification or detection of the S. aureus-specific sequence discriminates the presence of S. aureus from many other Staphylococci, e.g., Staphylococcus arlettae; Staphylococcus auricularis; Staphylococcus capitis; Staphylococcus caprae; Staphylococcus carnosus; Staphylococcus chromogenes; Staphylococcus cohnii subsp. Urealyticum; Staphylococcus delphini; Staphylococcus epidermidis (MRSE); Staphylococcus equorum; Staphylococcus felis; Staphylococcus gallinarum; Staphylococcus haemolyticus; Staphylococcus hominis; Staphylococcus intermedius; Staphylococcus kloosii; Staphylococcus lentus; Staphylococcus pasteuri; Staphylococcus pulvereri; Staphylococcus saprophyticus; Staphylococcus sciuri; Staphylococcus simulans; Staphylococcus warneri; and/or Staphylococcus xylosus. In some embodiments, amplification or detection of the S. aureus-specific sequence discriminates the presence of S. aureus from CNS. Optionally, amplification or detection of the S. aureus-specific sequence can be highly specific for methicillin-resistant S. aureus, so that nucleic acids from no other known organisms are detected.

In some embodiments, one or more oligomers in a set, kit, composition, or reaction mixture comprise a methylated cytosine (e.g., 5-methylcytosine). In some embodiments, at least about half of the cytosines in an oligomer are methylated. In some embodiments, all or substantially all (e.g., all but one or two) of the cytosines in an oligomer are methylated (e.g., one or more cytosines at the 3′ end or within 2, 3, 4, or 5 bases of 3′ end are unmethylated).

In some embodiments, an oligomer is provided that comprises a label and/or is non-extendable. Such an oligomer can be used as a probe or as part of a probe system (e.g., as a FRET cassette in combination with a target-binding detection oligomer). In some embodiments, the label is a non-nucleotide label. Suitable labels include compounds that emit a detectable light signal, such as fluorophores or luminescent (e.g., chemiluminescent) compounds that can be detected in a homogeneous mixture. More than one label, and more than one type of label, can be present on a particular probe, or detection can rely on using a mixture of probes in which each probe is labeled with a compound that produces a detectable signal (see for example, U.S. Pat. Nos. 6,180,340 and 6,350,579). Labels can be attached to a probe by various means including covalent linkages, chelation, and ionic interactions. In some embodiments the label is covalently attached. For example, in some embodiments, a detection probe has an attached chemiluminescent label such as an acridinium ester (AE) compound (see for example, U.S. Pat. Nos. 5,185,439, 5,639,604, 5,585,481, and 5,656,744). A label, such as a fluorescent or chemiluminescent label, can be attached to the probe by a non-nucleotide linker (see for example, U.S. Pat. Nos. 5,585,481, 5,656,744, and 5,639,604). In some embodiments, an oligomer is provided that is non-extendible and hybridizes to a site in a target nucleic acid that overlaps the hybridization site of an additional oligomer in a kit or composition, such as an amplification oligomer, or an oligomer that is chemically modified to preclude extension. Hybridization of such oligomers can form a substrate for a structure-specific nuclease, for example, as part of the detection mechanism in an INVADER® or INVADER PLUS® assay.

In some embodiments, a labeled oligomer (e.g., comprising a fluorescent label) further comprises a second label that interacts with the first label. For example, the second label can be a quencher. Such probes can be used (e.g., in TAQMAN® assays) where hybridization of the probe to a target or amplicon followed by nucleolysis by a polymerase comprising 5′-3′ exonuclease activity results in liberation of the fluorescent label and thereby increased fluorescence, or fluorescence independent of the interaction with the second label. FRET cassettes useful in INVADER® or INVADER PLUS® assays are other examples of dual-labeled probes that include a fluorophore and a quencher.

In some applications, one or more probes exhibiting at least some degree of self-complementarity can be used to facilitate detection of probe: target duplexes in a test sample without first requiring the removal of unhybridized probe prior to detection. Specific embodiments of such detection probes include, for example, probes that form conformations held by intramolecular hybridization, such as conformations generally referred to as hairpins. Suitable hairpin probes include a “molecular torch” (see U.S. Pat. Nos. 6,849,412, 6,835,542, 6,534,274, and 6,361,945) and a “molecular beacon” (see U.S. Pat. Nos. 5,118,801 and 5,312,728). Molecular torches include distinct regions of self-complementarity (coined “the target binding domain” and “the target closing domain”) which are connected by a joining region (e.g., a —(CH₂CH₂O)₃— linker) and which hybridize to one another under predetermined hybridization assay conditions. When exposed to an appropriate target or denaturing conditions, the two complementary regions (which can be fully or partially complementary) of the molecular torch melt, leaving the target binding domain available for hybridization to a target sequence when the predetermined hybridization assay conditions are restored. Molecular torches are designed so that the target binding domain favors hybridization to the target sequence over the target closing domain. The target binding domain and the target closing domain of a molecular torch include interacting labels (e.g., fluorophore and quencher) positioned so that a different signal is produced when the molecular torch is self-hybridized as opposed to when the molecular torch is hybridized to a target nucleic acid, thereby permitting detection of probe: target duplexes in a test sample in the presence of unhybridized probe having a viable label associated therewith.

Examples of interacting donor/acceptor label pairs that can be used in connection with the disclosure include fluorescein/tetramethylrhodamine, IAEDANS/fluororescein, EDANS/DABCYL, coumarin/DABCYL, fluorescein/fluorescein, BODIPY® FL/BODIPY® FL, fluorescein/DABCYL, lucifer yellow/DABCYL, BODIPY®/DABCYL, eosine/DABCYL, erythrosine/DABCYL, tetramethylrhodamine/DABCYL, TEXAS RED®/DABCYL, CY5/BHQ1®, CY5/BHQ2®, CY3/BHQI®, CY3/BHQ2® and fluorescein/QSY7® dye. Those having an ordinary level of skill in the art will understand that when donor and acceptor dyes are different, energy transfer can be detected by the appearance of sensitized fluorescence of the acceptor or by quenching of donor fluorescence. Non-fluorescent acceptors such as DABCYL and the QSY7® dyes advantageously eliminate the potential problem of background fluorescence resulting from direct (i.e., non-sensitized) acceptor excitation. Exemplary fluorophore moieties that can be used as one member of a donor-acceptor pair include fluorescein, HEX, ROX, and the CY dyes (such as CY5). Exemplary quencher moieties that can be used as another member of a donor-acceptor pair include DABCYL. The BLACKBERRY QUENCHER® and BLACK HOLE QUENCHER® moieties, both of which are available from Biosearch Technologies, Inc., (Novato, Calif.). One of ordinary skill in the art will be able to use appropriate pairings of donor and acceptor labels for use in various detection formats (e.g., FRET, TAQMAN®, INVADER®, etc.).

In some embodiments, a detection oligomer (e.g., probe, primary probe, or labeled probe) is non-extendable. For example, the labeled oligomer can be rendered non-extendable by a 3′-adduct (e.g., 3′-phosphorylation or 3′-alkanediol), having a 3′-terminal 3′-deoxynucleotide (e.g., a terminal 2′,3′-dideoxynucleotide), having a 3′-terminal inverted nucleotide (e.g., in which the last nucleotide is inverted such that it is joined to the penultimate nucleotide by a 3′ to 3′ phosphodiester linkage or analog thereof, such as a phosphorothioate), or having an attached fluorophore, quencher, or other label that interferes with extension (possibly but not necessarily attached via 3′ position of the terminal nucleotide). In some embodiments, 3′-terminal nucleotide is not methylated. In some embodiments, a detection oligomer comprises a 3′-terminal adduct such as a 3′-alkanediol (e.g., hexanediol). In some embodiments, a detection oligomer is configured to specifically hybridize to a target nucleic acid amplicon (e.g., the oligomer comprises or consists of a target-hybridizing sequence sufficiently complementary to the amplicon for specific hybridization).

Also provided by the disclosure is a reaction mixture for determining the presence or absence of a target nucleic acid in a sample. A reaction mixture in accordance with the present disclosure comprises a primer set comprising at least one dual-specificity primer as described herein for amplification of a target nucleic acid sequence; and optionally a detection probe oligomer as described herein for determining the presence or absence of an amplicon. The reaction mixture can further include a number of optional components such as, for example, capture probes (e.g., poly-(k) capture probes as described in US Pat. App. Pub. No. 2013/0209992). Amplification reaction mixtures typically will include other reagents suitable for performing in vitro amplification such as, buffers, salt solutions, appropriate nucleotide triphosphates (e.g., dATP, dCTP, dGTP, and one or both of dTTP or dUTP; and/or ATP, CTP, GTP and UTP), and/or enzymes (e.g., a thermostable DNA polymerase, and/or reverse transcriptase and/or RNA polymerase and/or FEN enzyme), and will typically include test sample components in which a target nucleic acid may or may not be present.

A reaction mixture can include amplification oligomers for at least one target nucleic acid. For example, the reaction mixture can include amplification oligomers for target nucleic acids such as the orfX/SCCmec junction (e.g., including multiple types thereof as discussed above), mecA and/or mecC, and an S. aureus-specific sequence such as a GAPDH sequence. In addition, for a reaction mixture that includes a detection probe together with an amplification oligomer combination, selection of amplification oligomers and detection probe oligomers for a reaction mixture are linked by a common target region (i.e., the reaction mixture will include a probe that binds to a sequence amplifiable by an amplification oligomer combination of the reaction mixture).

Also provided by the subject disclosure are kits for practicing the methods described herein. A kit in accordance with the present disclosure includes a primer set with at least one dual-specificity primer for amplification of a target nucleic acid sequence; and optionally at least one detection probe oligomer as described herein for determining the presence or absence of an amplicon. In some embodiments, any oligomer combination described herein is present in the kit. The kits can further include a number of optional components such as, for example, capture probes (e.g., poly-(k) capture probes as described in US Pat. App. Pub. No. 2013/0209992).

Other reagents that can be present in the kits include reagents suitable for performing in vitro amplification such as buffers, salt solutions, appropriate nucleotide triphosphates (e.g., dATP, dCTP, dGTP, and one or both of dTTP or dUTP; and/or ATP, CTP, GTP and UTP), and/or enzymes (e.g., a thermostable DNA polymerase, and/or reverse transcriptase and/or RNA polymerase and/or FEN enzyme), and will typically include test sample components, in which a target nucleic acid (e.g., MRSA target nucleic acid) may or may not be present. A kit can include amplification oligomers for at least one target nucleic acid, such as the orfX/SCCmec junction (e.g., including multiple types thereof as discussed above), mecA and/or mecC, and an S. aureus-specific sequence or a sequence indicative of S. aureus such as a GAPDH sequence. In addition, for a kit that includes a detection probe together with an amplification oligomer combination, selection of amplification oligomers and detection probe oligomers for a reaction mixture are linked by a common target region (i.e., the reaction mixture will include a probe that binds to a sequence amplifiable by an amplification oligomer combination of the reaction mixture). In certain embodiments, the kit further includes a set of instructions for practicing methods in accordance with the present disclosure, where the instructions can be associated with a package insert and/or the packaging of the kit or the components thereof.

The methods described herein are to be understood as teaching use(s) of the disclosed reagents for the purpose of achieving an intended result. Any of the oligomers and any combinations (e.g., kits and compositions, including but not limited to reaction mixtures) comprising such an oligomer are to be understood as also disclosed for use in detecting or quantifying target nucleic acid.

Broadly speaking, methods can comprise one or more of the following: target capture, in which target nucleic acid (e.g., from a sample, such as a clinical sample) is annealed to a capture oligomer (e.g., a specific or nonspecific capture oligomer); isolation (e.g., washing, to remove material not associated with a capture oligomer); amplification; and amplicon detection, which for example can be performed in real-time with amplification. Certain embodiments involve each of the foregoing steps. Certain embodiments involve exponential amplification, optionally with a preceding linear amplification step, as illustrated by Nelson et al., in U.S. Pat. No. 10,196,674. In accordance with Nelson et al., linear amplification of template nucleic acids can be accomplished using target-capture and washing steps to remove unhybridized dual-specificity primers prior to any polymerization reaction, thereby limiting use of that primer to a single round of extension. Certain embodiments involve exponential amplification and amplicon detection. Certain embodiments involve any two of the components listed above. Certain embodiments involve any two components listed adjacently above (e.g., washing and amplification, or amplification and detection).

In some embodiments, amplification comprises (1) contacting the sample with at least two oligomers for amplifying a target nucleic acid, where the oligomers include at least two amplification oligomers as described above (e.g., one or more oriented in the sense direction and one or more oriented in the antisense direction for exponential amplification); (2) performing an in vitro nucleic acid amplification reaction, where any target nucleic acid present in the sample is used as a template for generating an amplification product; and (3) detecting the presence or absence of the amplicon, thereby determining the presence or absence of the target nucleic acid sequences in the sample.

Optionally, only the dual-specificity primer is extended by a polymerase in the amplification reaction. Optionally, the reaction that amplifies a sequence from a target nucleic acid includes only a single dual-specificity primer to participate in amplification of the target nucleic acid. Optionally, the amplification reaction includes only a single primer (i.e., the dual-specificity primer). Optionally, the single-primer amplification reaction further includes an oligonucleotide that is blocked from extension at its 3′-end.

A detection method in accordance with the present disclosure can further include the step of obtaining the sample to be subjected to subsequent steps of the method. In certain embodiments, “obtaining” a sample to be used includes, for example, receiving the sample at a testing facility or other location where one or more steps of the method are performed, and/or retrieving the sample from a location (e.g., from storage or other depository) within a facility where one or more steps of the method are performed.

In certain embodiments, the method includes purifying the target nucleic acid from other components (e.g., non-nucleic acid components) in a sample before an amplification (e.g., a capture step). Such purification can include methods of separating and/or concentrating organisms contained in a sample from other sample components, or removing or degrading non-nucleic acid sample components (e.g., protein, carbohydrate, salt, lipid, etc.). In some embodiments, RNA in the sample is degraded (e.g., with RNase and/or heat), and optionally the RNase is removed or inactivated and/or degraded RNA is removed.

In some embodiments, purifying the target nucleic acid includes capturing the target nucleic acid to specifically or non-specifically separate the target nucleic acid from other sample components. Non-specific target capture methods can involve selective precipitation of nucleic acids from a substantially aqueous mixture, adherence of nucleic acids to a support that is washed to remove other non-nucleic acid sample components, or other means of physically separating nucleic acids from a mixture that contains target nucleic acid and other sample components.

Target capture can occur in a solution phase mixture that contains one or more capture probe oligomers that hybridize to the target nucleic acid sequence under hybridizing conditions. For embodiments comprising a capture probe tail, the target:capture-probe complex is captured by applying hybridization conditions so that the capture probe tail hybridizes to the immobilized probe. Certain embodiments use a particulate solid support, such as paramagnetic beads.

Isolation can follow capture, where the complex on the solid support is separated from other sample components. Isolation can be accomplished by any appropiate technique, such as washing a support associated with the target nucleic acid sequence one or more times to remove other sample components and/or unbound oligomer. In embodiments using a particulate solid support, such as paramagnetic beads, particles associated with the target nucleic acid sequence can be suspended in a washing solution and retrieved from the washing solution, in some embodiments by using magnetic attraction. Examples of nucleic acid enrichment or purification by this target capture method are disclosed by Weisburg et al., in U.S. Pat. No. 6,110,678. To limit the number of handling steps, the target nucleic acid sequence can be amplified by simply mixing the target nucleic acid sequence in the complex on the support with amplification oligomers and proceeding with amplification steps.

Exponentially amplifying a target nucleic acid sequence utilizes an in vitro amplification reaction using at least two amplification oligomers as described herein, where at least one of the amplification oligomers is a dual-specificity primer. In some embodiments, at least two amplification oligomers as described above are provided. In some embodiments, a plurality of pairs of amplification oligomers are provided, where the plurality comprises oligomer pairs configured to hybridize to MREJ sequences of at least 2, 3, 4, 5, 6, 7, 8, 9, 10, 11, 12, 13, or more types of MRSA MREJ sequences. In some embodiments, such types of MRSA MREJ sequences include at least 2, 3, 4, 5, 6, 7, 8, 9, 10, 11, 12, 13, or 14 of MREJ types i, ii, iii, iv, v, vi, vii, viii, ix, xii, xiii, xiv, xv, or xxi. The amplification reaction can be either temperature cycled or isothermal. Suitable amplification methods include, for example, replicase-mediated amplification, polymerase chain reaction (PCR), ligase chain reaction (LCR), strand-displacement amplification (SDA), and transcription-mediated amplification (TMA).

A detection step can be performed using any of a variety of known techniques to detect a signal specifically associated with the amplified target sequence, such as by hybridizing the amplification product with a labeled detection probe and detecting a signal resulting from the labeled probe (including from label released from the probe following hybridization in some embodiments). Preferred detectable labels include fluorescent moieties. In some embodiments, the labeled probe includes a second moiety, such as a quencher or other moiety that interacts with the first label, as discussed above. The detection step can also provide additional information on the amplified sequence, such as all or a portion of its nucleic acid sequence.

Detection can be performed after the amplification reaction is completed, or can be performed concurrent with amplifying the target region (e.g., in real-time). In one embodiment, the detection step allows homogeneous detection (e.g., detection of the hybridized probe without removal of unhybridized probe from the mixture (see U.S. Pat. Nos. 5,639,604 and 5,283,174)). In some embodiments, the nucleic acids are associated with a surface that results in a physical change, such as a detectable electrical change. Amplified nucleic acids can be detected by concentrating them in or on a matrix and detecting the nucleic acids or dyes associated with them (e.g., an intercalating agent such as ethidium bromide, or SYBR Green dye (Thermo Fisher, Inc.; Waltham, MA)), or detecting an increase in dye associated with nucleic acid in solution phase. Other methods of detection can use nucleic acid detection probes that are configured to specifically hybridize to a sequence in the amplified product and detecting the presence of the probe:product complex, or by using a complex of probes that can amplify the detectable signal associated with the amplified products (e.g., U.S. Pat. Nos. 5,424,413, 5,451,503, and 5,849,481). Directly or indirectly labeled probes that specifically associate with the amplicon provide a detectable signal that indicates the presence of the target nucleic acid in the sample. In some embodiments, the amplified product will contain a target sequence in or complementary to a sequence in the MRSA chromosome, and a probe will bind directly or indirectly to a sequence contained in the amplicon to indicate the presence of MRSA nucleic acid in the tested sample.

In embodiments that detect the amplicon near or at the end of the amplification step, a linear detection probe can be used to provide a signal to indicate hybridization of the probe to the amplicon. One example of such detection uses a chemiluminescently labeled probe that hybridizes to target nucleic acid sequence. In some embodiments, the chemiluminescent label is then hydrolyzed from non-hybridized probe. Detection of the surviving chemiluminescent label is performed using a luminometer (see, e.g., International Pub. No. WO 89/002476). In other embodiments that use real-time detection, the detection probe can be a hairpin probe such as a molecular beacon or a molecular torch that is labeled with a reporter moiety that is detected when the probe binds to amplified product. Alternatively, hydrolysis probes including fluorophore and quencher moieties in energy transfer relationship also can be used. Such probes can comprise target-hybridizing sequences and non-target-hybridizing sequences. Various forms of such probes are described, for example, in U.S. Pat. Nos. 5,118,801, 5,312,728, 5,925,517, 6,150,097, 6,849,412, 6,835,542, 6,534,274, and 6,361,945; and US Pat. App. Pub. Nos. 2006/0068417A1 and 2006/0194240A1.

In some embodiments, amplicon is detected through an invasive cleavage assay that provides means for forming an invasive cleavage structure that requires the presence of a target nucleic acid sequence. The assay further involves cleaving the invasive cleavage structure to release distinctive cleavage products. A cleavage agent such as a FEN enzyme, for example, is used to cleave the target-dependent invasive cleavage structure, thereby resulting in cleavage products that indicate the presence of specific target nucleic acid sequences in the sample. When two oligonucleotides hybridize to a target nucleic acid sequence such that they form an overlapping invasive cleavage structure, as defined above, invasive cleavage can occur. Through the interaction of a cleavage agent (e.g., FEN enzyme) and the upstream oligonucleotide (i.e., the invasive probe), the cleavage agent can be made to cleave the downstream oligonucleotide (i.e., the primary probe) at an internal site such that a distinctive fragment is produced. The fragment, sometimes referred to as a “liberated flap” or “cleaved 5′ flap” can then interact with a secondary probe, such as a FRET cassette (e.g., by participating as an invasive probe in a subsequent reaction that generates a fluorescent signal). Such embodiments are described in U.S. Pat. Nos. 5,846,717, 5,985,557, 5,994,069, 6,001,567, and 6,090,543; International Pub. Nos. WO 97/27214, WO 98/42873, and WO 2016/179093; Lyamichev et al., Nat. Biotech., 17:292 (1999); and Hall et al., PNAS, 97:8272 (2000). More specifically, a plurality of INVADER® reactions (e.g., combined in a single reaction mixture), can be used for the multiplex applications disclosed herein, including detection of various orfX-SCCmec junctions, mecA, mecC, an S. aureus-specific gene, and/or an internal control.

Invasive cleavage assays can be used for detecting specific target sequences in unamplified, as well as amplified DNA (e.g., PCR product(s)), including genomic DNA, RNA, or an amplicon thereof. The primary probe and the invasive probe hybridize in tandem to the target nucleic acid sequence to form an overlapping structure. An unpaired “flap” is included on 5′ end of the primary probe. A clevage agent (e.g. a FEN enzyme, such as the Cleavase® enzymes available from Hologic, Inc.) recognizes the overlap and cleaves off the unpaired 5′ flap. In some embodiments, in a secondary reaction, this cleaved product serves as an invasive probe on a FRET cassette to again create a structure recognized by the structure-specific enzyme. When the two labels on a single FRET cassette are separated by cleavage, a detectable fluorescent signal above background fluorescence is produced. Consequently, cleavage of this second invasive cleavage structure results in an increase in fluorescence, indicating the presence of the target nucleic acid sequence.

In some embodiments, one or more of an internal amplification control polynucleotide (“internal control”; e.g., a plasmid, plasmid fragment, or other polynucleotide, preferably with a sequence unrelated to target nucleic acid sequences, for example, to orfX, MREJ, mecA, mecC, and an S. aureus-specific or S. aureus-indicative gene), and oligomers for amplifying and detecting the internal control are provided as components of a composition or kit disclosed herein and/or are used in a method disclosed herein. Detection of an amplicon from the internal control can serve to avoid false negatives due to instrument or reagent failures when no target sequences are detected.

The following Examples are provided to illustrate certain disclosed embodiments, and should not be construed as limiting the scope of this disclosure in any way.

Example 1 describes procedures that demonstrated how dual-specificity primers were used to achieve target-specific amplification and detection. Template sequences in the nucleic acid amplification reactions corresponded either to sequences present at the interrupted orfX insertion junction of methicillin-resistant S. aureus (MRSA), or to sequences characteristic of wild-type methicillin-sensitive S. aureus (MSSA). In this procedure, 5′ sequences of the dual-specificity primers hybridized to a target nucleic acid sequence that differed between MRSA and MSSA, while 3′ sequences of the dual-specificity primers hybridized to target nucleic acid sequences that were substantially common to both MRSA and MSSA. Results obtained using two different dual-specificity primers and one standard amplification primer were compared.

### Example 1

### Demonstrating Features of Dual-Specificity Primers

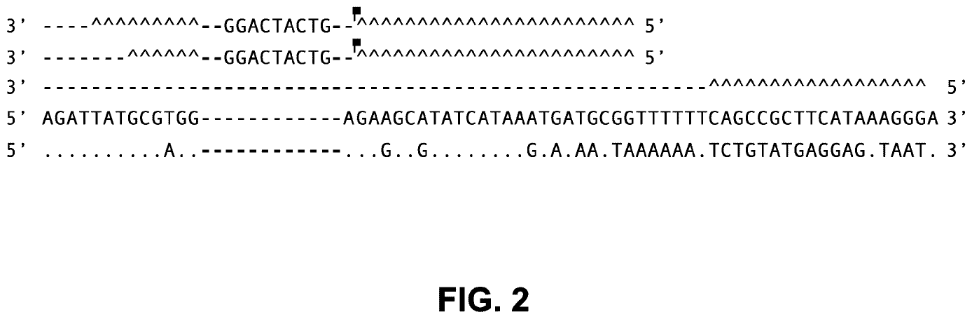

Parallel reactions were prepared to amplify two different template nucleic acids by the polymerase chain reaction using one of three different reverse primers with invasive cleavage (e.g., “Invader”) detection of amplicons. Template nucleic acids represented either MSSA (i.e., including the relevant sequence of SEQ ID NO:11) or MRSA (i.e., including the relevant sequence of SEQ ID NO:12). Input template concentrations used in the amplification reactions ranged from 102 copies/ml (“CAL01”) up to 106 copies/ml (“CAL 05”). Negative control reactions omitted template nucleic acid. Three different reverse primers were used, each separate from the other. The first reverse primer had the sequence of SEQ ID NO:1 (i.e., a standard primer that was fully complementary to the MRSA template of SEQ ID NO:12, but not fully complementary to the MSSA template of SEQ ID NO:11). The second reverse primer was a dual-specificity primer and had the sequence of PRIMER 2 (i.e., a dual-specificity primer having a 23 nucleotide 5′-end insert-binding sequence and 6 nucleotide 3′-end target-binding sequence) (SEQ ID NOS: 2 and 14). The third reverse primer was also a dual-specificity primer and had the sequence of PRIMER 3 (i.e., a dual-specificity primer having a 23 nucleotide 5′-end insert-binding sequence and 9 nucleotide 3′-end target-binding sequence) (SEQ ID NOS: 3 and 15). The two dual-specificity primers differed from each other only in the length of the 3′-end target-binding sequence, where one primer was 3 bases longer than the other. While the standard primer used for comparison did not include any template termination moiety, each dual-specificity primer included a non-nucleotide linker (e.g. a C9 spacer serving as the template termination moiety) between nucleotide positions 23 and 24. Sequences of the standard reverse primer, the dual-specificity primers, and the relevant template sequences are aligned in FIG. 2. The forward primer used in the amplification reactions had the sequence of SEQ ID NO:8. The primary probe (subject to cleavage by the forward primer during amplification reactions) had the sequence of SEQ ID NO:9, and the FRET cassette had the sequence of SEQ ID NO: 10.

Amplicons synthesized using the three different reverse primers were detected using the invasive cleavage system with real-time fluorescence monitoring. In addition to primer and probe oligonucleotides, all reaction mixtures included an aqueous mixture of a pH buffer, salts, dNTPs, a flap endonuclease enzyme, and a reverse transcriptase able to replicate DNA templates. The flap endonuclease was included to facilitate invasive cleavage reactions used for detection of amplification products. Reactions detecting amplicons using a different probe system (e.g., hydrolysis probes, molecular beacons, molecular torches, etc.) would not use this enzyme. Amplification reactions monitored using real-time detection of fluorescent signals were carried out using standard laboratory techniques familiar to those having an ordinary level of skill in the art.

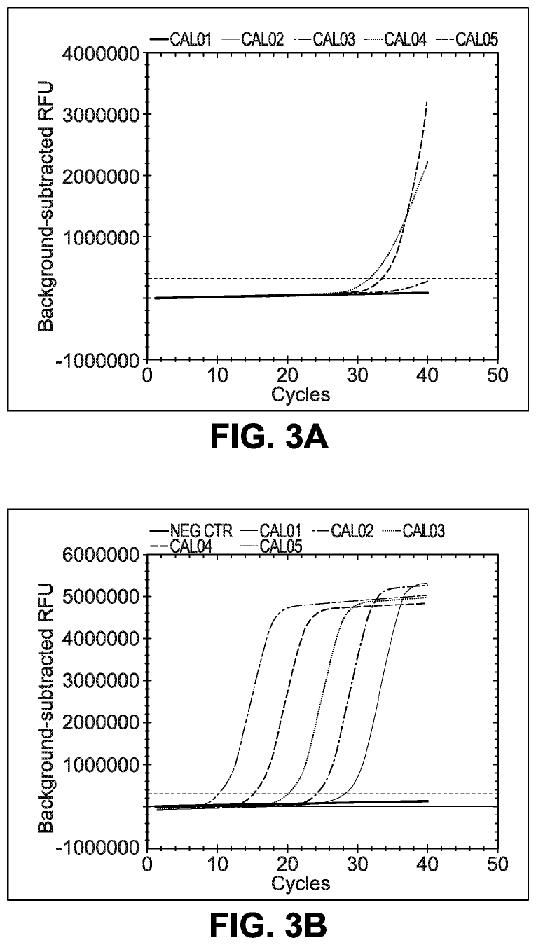

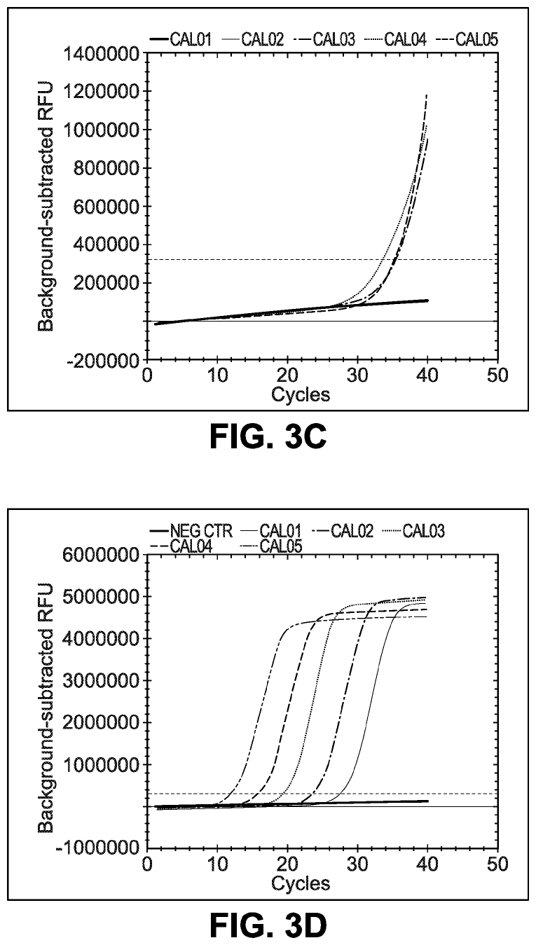

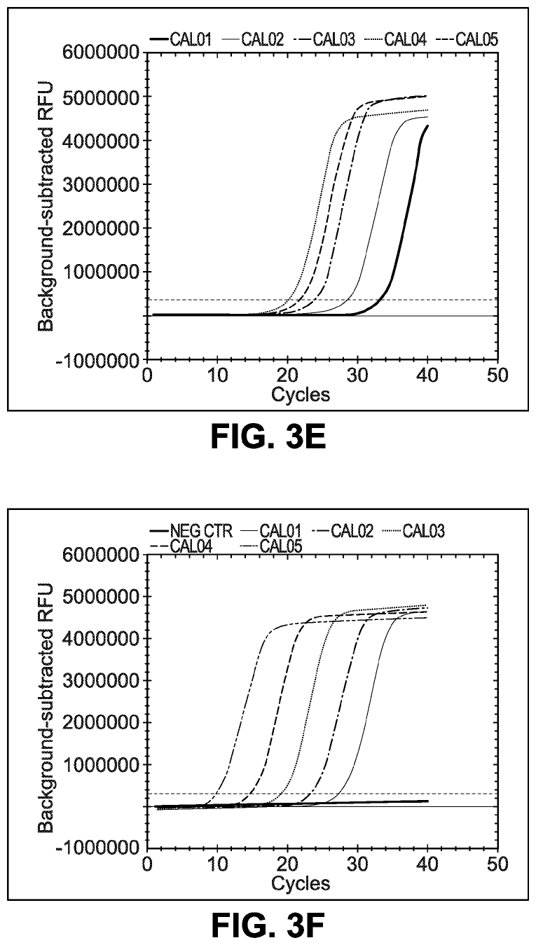

Results of the procedure are illustrated in FIGS. 3A-3F, and in Table 1. FIGS. 3A, 3C and 3E present results obtained using MSSA nucleic acid templates. FIGS. 3B, 3D and 3F present results obtained using MRSA nucleic acid templates that included a junction between the chromosomal orfX gene and an insert sequence carrying a gene responsible for methicillin resistance. The plotted real-time run curves present measured fluorescence as a function of cycle number for all 40 reaction cycles in the procedure. The horizontal line just above 300,000 RFU (relative fluorescence units) in each of the plots represented the threshold level of amplification for determining TCycles (e.g., an indication of a predetermined level of reaction progress). The first 32 cycles of amplification reactions were typically regarded as sufficient for detecting low levels of the target analytes, because higher cycle numbers can be associated with artifacts that did not reflect specific target amplification.

Run curves shown in FIGS. 3A and 3B represent results from reactions performed using the standard reverse primer of SEQ ID NO:1 that was specific for the insert encoding methicillin resistance. Run curves shown in FIGS. 3C and 3D represent results from reactions performed using the dual-specificity primer of PRIMER 2. Run curves shown in FIGS. 3E and 3F represent results from reactions performed using the dual-specificity primer of PRIMER 3, that was three nucleotides longer than the sequence of PRIMER 2.

The reverse primer of SEQ ID NO: 1 was fully complementary to the MRSA template nucleic acid amplified in the reaction yielding results presented in FIG. 3B, but was complementary to only 15 out of 23 bases of the MSSA template nucleic acid amplified in the reaction yielding results presented in FIG. 3A. As indicated in FIG. 2, 3′ portions of 5′-end insert-binding sequences of the dual-specificity primers used in the procedure were complementary to 14 of 18 bases of the MSSA template of SEQ ID NO:11.

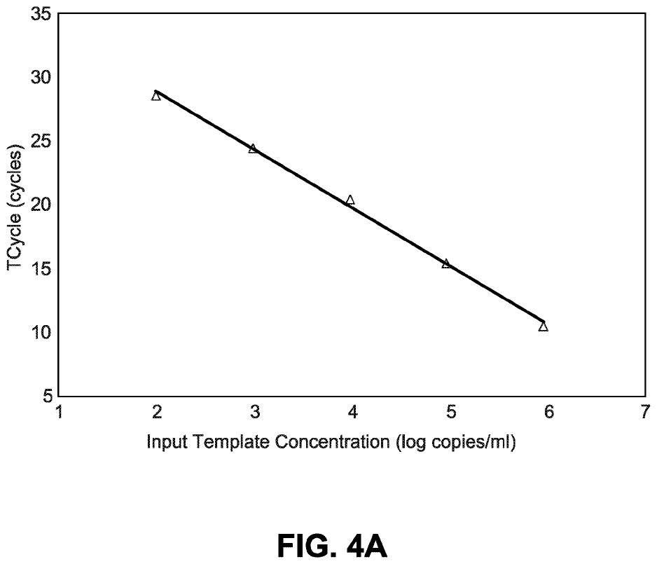

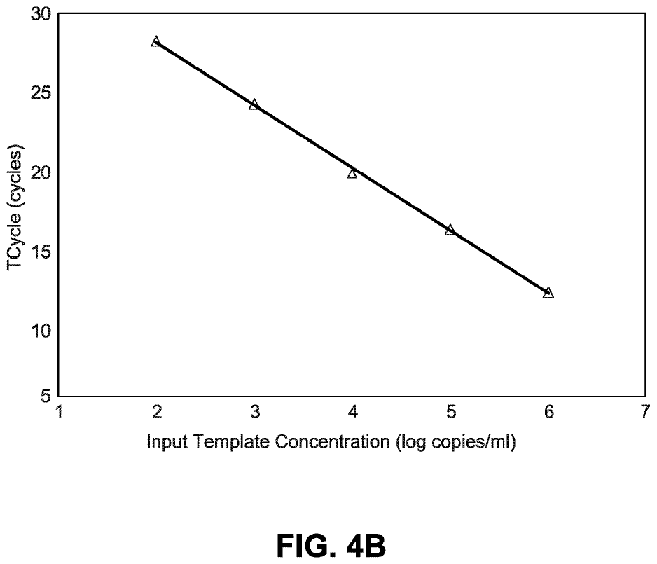

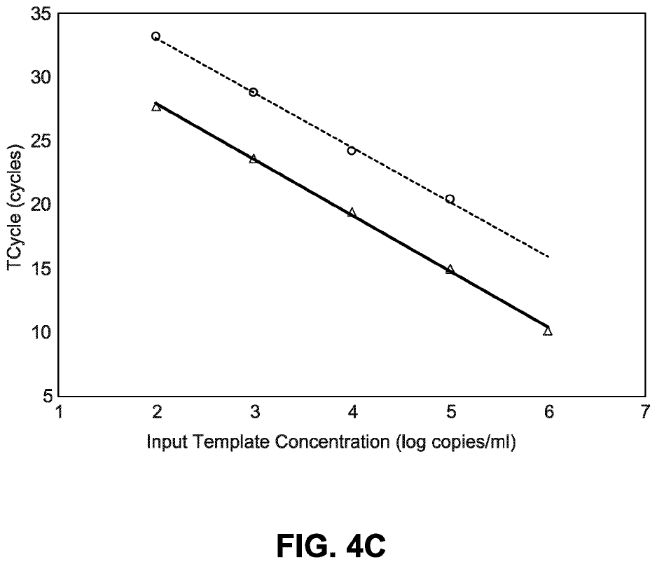

The dual-specificity primer of PRIMER 2 included a 5′-end insert-binding sequence that was 23 bases long, and a 3′-end target-binding sequence that was 6 bases long. Template nucleic acids used in the reactions of FIGS. 3C and 3D were fully complementary to the sequence of 3′-end target-binding sequence (FIG. 3D), or mismatched at a single base position (FIG. 3C). Fluorescent signals detected in reactions conducted using highly concentrated template nucleic acid in the reactions of FIG. 3C exhibited TCycles occurring later than 32 cycles, and so were not regarded as reliably indicating the presence or amount of the MSSA template nucleic acid. Conversely, fluorescent signals detected in reactions conducted using all concentrations of template nucleic acid in the reactions of FIG. 3D exhibited TCycles occurring earlier than 32 cycles, and were useful for quantifying the MRSA template (see FIG. 4).

The dual-specificity primer of PRIMER 3 included the same 5′-end insert-binding sequence (23 bases long) as dual-specificity primer of PRIMER 2, but had a 3′-end target-binding sequence that was 9 bases long (i.e., 3 bases longer than 3′-end target-binding sequence of dual-specificity primer of PRIMER 2). As indicated in FIG. 2, template nucleic acids used in the reactions yielding results shown in FIGS. 3E and 3F were fully complementary to the sequence of 3′-end target-binding sequence (FIG. 3F), or mismatched at a single base position (FIG. 3E). The 3′-end target-binding sequence of PRIMER 3 permitted amplification of both the MSSA and MRSA nucleic acid templates, and so did not qualitatively distinguish the templates from each other. On the other hand, reducing the length of 3′-end target-binding sequence permitted the dual-specificity primer of PRIMER 2 to discriminate between the MSSA and MRSA nucleic acid templates, which differed by only a single base in the sequence hybridized by 3′-end target-binding sequence of the dual-specificity primer, but also differed in the target sequence that would be hybridized by 5′-end insert-binding sequence of the dual-specificity primer. Thus, 3′-end target-binding sequence of the dual-specificity primer contributed to specificity in priming and amplification reactions. While not wishing to be bound by any particular theory of operation, it is possible that the primer of PRIMER 3 initiated the amplification reaction on the MSSA template without hybridization of the 5′-end insert-binding sequence to the target nucleic acid. Alternatively, hybridization of the 5′-end insert-binding sequence to the template nucleic acid promoted interaction of 3′ target-binding sequence of the dual-specificity primer and the mismatched template nucleic acid. Preferably, 3′-end sequence of the dual-specificity primer is shorter than 9 bases. Preferably, 3′-end sequence is 6-8 bases in length, more preferably 6 bases in length to achieve the desired amplification specificity requiring simultaneous hybridization of 5′-end sequence of the primer.

Table 1 presents TCycle values determined from the different run curves shown in FIGS. 3A-3F. Numerical values were not entered into the table for reactions conducted using the MSSA nucleic acid template comprising the sequence of SEQ ID NO:11 and either the standard primer of SEQ ID NO: 1 or the dual-specificity primer PRIMER 2 if the combinations of primers and templates did not exhibit clear evidence for specific amplification. A preferred criterion for specific amplification involved determination of a TCycle value less than a predetermined threshold cycle number for amplification of a specified input concentration of template. For example, specific amplification was determined as reflecting a TCycle value of 32 cycles or less for amplification of a template at an input concentration of 100 copies/ml (i.e., CAL01).

TABLE 1
TCycle Values Determined Using Two Different Primers

| | | | Log | |
|---|---|---|---|---|
| | | Sample | Target | |
| Primer | Template | Type | Conc. | TCycle |
| Standard Primer | MRSA | Neg Control | None | |
| SEQ ID NO: 1 | SEQ ID NO: 12 | CAL01 | 2 | 28.65 |
| | | CAL02 | 3 | 24.57 |
| | | CAL03 | 4 | 20.60 |
| | | CAL04 | 5 | 15.60 |
| | | CAL05 | 6 | 10.68 |
| | MSSA | CAL01 | 2 | |
| | SEQ ID NO: 11 | CAL02 | 3 | |
| | | CAL03 | 4 | |
| | | CAL04 | 5 | |
| | | CAL05 | 6 | |
| Dual-Specificity | MRSA | Neg Control | None | |
| Primer | SEQ ID NO: 12 | CAL01 | 2 | 28.29 |
| PRIMER 2 (SEQ ID | | CAL02 | 3 | 24.34 |
| NOS: 2 and 14) | | CAL03 | 4 | 19.93 |
| | | CAL04 | 5 | 16.41 |
| | | CAL05 | 6 | 12.45 |
| | MSSA | CAL01 | 2 | |
| | SEQ ID NO: 11 | CAL02 | 3 | |
| | | CAL03 | 4 | |
| | | CAL04 | 5 | |
| | | CAL05 | 6 | |
| Dual-Specificity | MRSA | Neg Control | None | |
| Primer | SEQ ID NO: 12 | CAL01 | 2 | 27.81 |
| PRIMER 3 (SEQ ID | | CAL02 | 3 | 23.69 |
| NOS: 3 and 15) | | CAL03 | 4 | 19.45 |
| | | CAL04 | 5 | 15.05 |
| | | CAL05 | 6 | 10.16 |
| | MSSA | CAL01 | 2 | 33.20 |
| | SEQ ID NO: 11 | CAL02 | 3 | 28.82 |
| | | CAL03 | 4 | 24.29 |
| | | CAL04 | 5 | 20.44 |
| | | CAL05 | 6 | N/A |

FIG. 44-4C display plots of the data from Table 1 that represented TCycle values determined for amplification of the template nucleic acids, where TCycles were determined at 40 cycles or less for amplification of a template at an input concentration of 100 copies/ml. With the exception of a single aberrant point at the highest input concentration of MSSA template amplified using the dual-specificity primer PRIMER 3, all TCycle values presented in Table 1 gave linear plots with R² values greater than 0.99.

The preceding example showed how a dual-specificity primer amplified a target nucleic acid in a highly specific manner when the target nucleic acid included a junction that separated the two spaced-apart binding sites for the dual-specificity primer (i.e., 5′ insert-binding and the 3′ target-binding sequences). The following example illustrates how this amplification specificity was maintained when the spacing between nucleic acid sequences hybridized by the primer was varied.

Example 2 describes procedures that demonstrated the joining region of a dual-specificity primer advantageously accommodated variable spacing between the two sequences in the target nucleic acid that hybridized to the primer. This modeled a situation where the positioning of the two spaced-apart sequences in the target nucleic acid was imprecise, variable, or even unknown prior to performing the amplification reaction. As in Example 1, the dual-specificity primers in the following procedure included 5′ sequences complementary to a sequence in the target nucleic acid characteristic of MRSA but not MSSA, and a 3′ sequence complementary to a sequence that was substantially similar (i.e., common) for the two target nucleic acids.

### Example 2

### Dual-Specificity Primers Tolerate Variable Spacing Between Hybridized Sequences

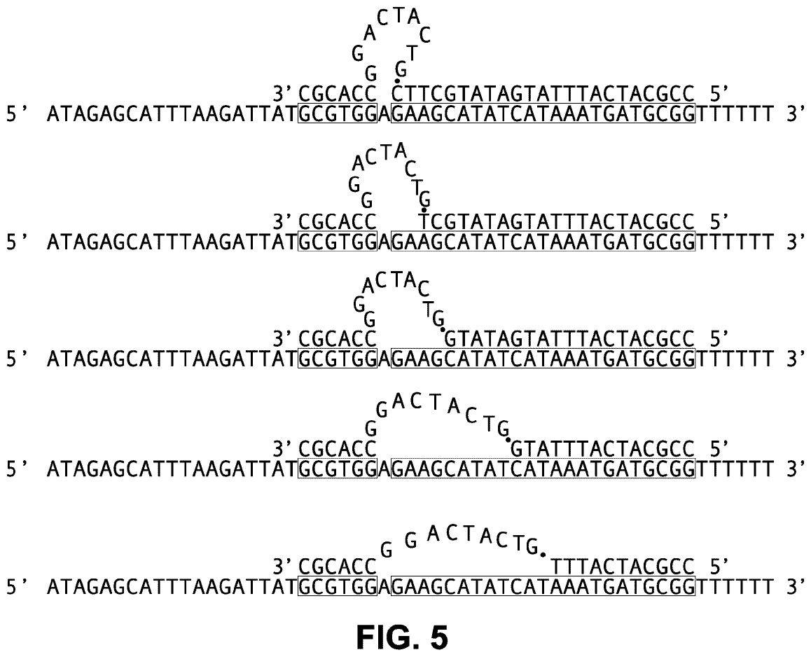

A collection of dual-specificity primers, each primer having a common sequence downstream of the template termination moiety, was synthesized using routine laboratory methods familiar to those having an ordinary level of skill in the art. Each primer among the collection further included a variably sized upstream sequence that was complementary to a target nucleic acid sequence within the MRSA target nucleic acid. More specifically, primer sequences upstream of the template termination moiety varied while the sequence at 5′-terminus remained fixed. As a result, the collection of primers shared identical 3′ sequences of nucleotides that hybridized to the target nucleic acid, and identical joining regions that included template termination moieties, but included upstream sequences that were different. Structures of the dual-specificity primers used in the procedure are presented in Table 2, with the template termination moiety being represented by the “\hat{ }” symbol. Positions where the primer and target nucleic acid did not hybridize by complementary base pairing, because the nucleotide was omitted from the primer sequence, are indicated by “=” symbols. A “-” symbol represents 3′-end of the junction sequence and 5′-end of the target-complementary sequence in the primer. Hybrid duplex structural interactions between dual-specificity primers and the target nucleic acid are diagrammed in FIG. 5.

TABLE 2
Dual-Specificity Primers Having Variable Spacing Between
First and Second Target-Hybridizing Sequences

| Dual-Specificity | |
|---|---|
| Primer | Sequence |
| PRIMER 2 (SEQ ID | 5′ CCGCATCATTTATGATATGCTTC^{∧}==GTCATCAGG-CCACGC 3′ |
| NOS: 2 and 14) | |
| PRIMER 4 (SEQ ID | 5′ CCGCATCATTTATGATATGCT^{∧}====GTCATCAGG-CCACGC 3′ |
| NOS: 4 and 14) | |
| PRIMER 5 (SEQ ID | 5′ CCGCATCATTTATGATATG^{∧}======GTCATCAGG-CCACGC 3′ |
| NOS: 5 and 14) | |
| PRIMER 6 (SEQ ID | 5′ CCGCATCATTTATG^{∧}===========GTCATCAGG-CCACGC 3′ |
| NOS: 6 and 14) | |
| PRIMER 7 (SEQ ID | 5′ CCGCATCATTT^{∧}==============GTCATCAGG-CCACGC 3′ |
| NOS: 7 and 14) | |

Amplification reactions were carried out essentially as described under Example 1, using the forward primer of SEQ ID NO:8 and target nucleic acids characteristic of MRSA (i.e., included junction sequence) or MSSA (i.e., did not include junction sequence). The dual-specificity primer of PRIMER 2 from Example 1 served as a control to indicate target-specific amplification. Fluorescent signals measured as the reaction was occurring were used to determine TCycle values using techniques that will be familiar to those having an ordinary level of skill in the art. Results from the procedure are presented in Table 3.

TABLE 3
Real-Time Amplification Results

| | | | Log Target | |
|---|---|---|---|---|
| Primer | Template | Sample Type | Conc. | TCycle |
| PRIMER 2 | Neg. CTRL | | 0 | |
| | MRSA | CAL 01 | 2.69 | 29.79 |
| | SEQ ID NO: 12 | CAL 02 | 3.69 | 27.04 |
| | | CAL 03 | 4.69 | 23.93 |
| | | CAL 04 | 5.69 | 20.64 |
| | | CAL 05 | 6.69 | 18.92 |
| | MSSA | CAL 01 | 2.69 | |
| | SEQ ID NO: 11 | CAL 02 | 3.69 | |
| | | CAL 03 | 4.69 | |
| | | CAL 04 | 5.69 | |
| | | CAL 05 | 6.69 | |
| PRIMER 4 | Neg. CTRL | | 0 | |
| | MRSA | CAL 01 | 2.69 | 30.01 |
| | SEQ ID NO: 12 | CAL 02 | 3.69 | 26.93 |
| | | CAL 03 | 4.69 | 23.72 |
| | | CAL 04 | 5.69 | 20.62 |
| | | CAL 05 | 6.69 | 18.98 |
| | MSSA | CAL 01 | 2.69 | |
| | SEQ ID NO: 11 | CAL 02 | 3.69 | |
| | | CAL 03 | 4.69 | |
| | | CAL 04 | 5.69 | |
| | | CAL 05 | 6.69 | |
| PRIMER 5 | Neg. CTRL | | 0 | |
| | MRSA | CAL 01 | 2.69 | 30.45 |
| | SEQ ID NO: 12 | CAL 02 | 3.69 | 27.21 |
| | | CAL 03 | 4.69 | 23.83 |
| | | CAL 04 | 5.69 | 20.75 |
| | | CAL 05 | 6.69 | 19.63 |
| | MSSA | CAL 01 | 2.69 | |
| | SEQ ID NO: 11 | CAL 02 | 3.69 | |
| | | CAL 03 | 4.69 | |
| | | CAL 04 | 5.69 | |
| | | CAL 05 | 6.69 | |
| PRIMER 6 | Neg. CTRL | | 0 | |
| | MRSA | CAL 01 | 2.69 | 30.39 |
| | SEQ ID NO: 12 | CAL 02 | 3.69 | 27.55 |
| | | CAL 03 | 4.69 | 24.41 |
| | | CAL 04 | 5.69 | 21.06 |
| | | CAL 05 | 6.69 | 20.39 |
| | MSSA | CAL 01 | 2.69 | |
| | SEQ ID NO: 11 | CAL 02 | 3.69 | |
| | | CAL 03 | 4.69 | |
| | | CAL 04 | 5.69 | |
| | | CAL 05 | 6.69 | |
| PRIMER 7 | Neg. CTRL | | 0 | |
| | MRSA | CAL 01 | 2.69 | 32.21 |
| | SEQ ID NO: 12 | CAL 02 | 3.69 | 29.20 |
| | | CAL 03 | 4.69 | 26.53 |
| | | CAL 04 | 5.69 | 23.35 |
| | | CAL 05 | 6.69 | 24.19 |
| | MSSA | CAL 01 | 2.69 | |
| | SEQ ID NO: 11 | CAL 02 | 3.69 | |
| | | CAL 03 | 4.69 | |
| | | CAL 04 | 5.69 | |
| | | CAL 05 | 6.69 | |

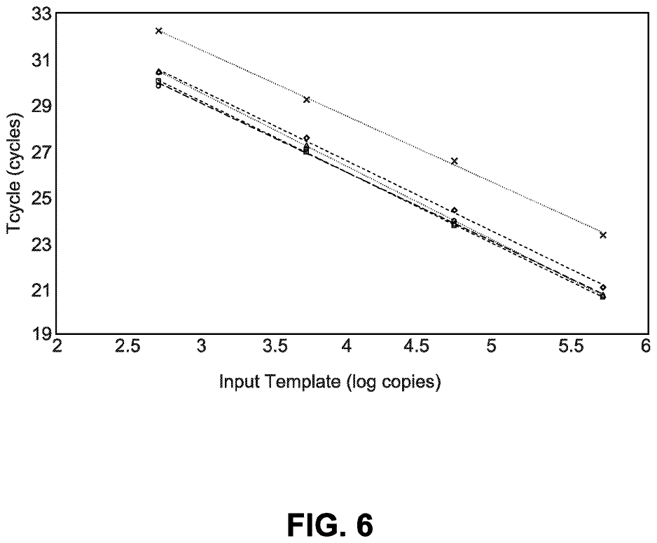

Results presented in Table 3 confirmed that each of the tested dual-specificity primers initiated amplification reactions that required the joining of first and second primer-hybridizing sequences in the target nucleic acid sequence. Amplification was not observed in trials conducted using the MSSA template nucleic acid. All of the tested dual-specificity primers having 3′ target-binding sequences 6 nucleotides long exhibited very specific amplification characteristics. Tabulated results for the first four calibrators in each trial from Table 3 are graphically presented in FIG. 6.

The observed trend toward increasing TCycle values at fixed target input levels as the primer sequence upstream of the template termination moiety was progressively shortened confirmed the critical nature of 5′ insert-binding sequence for initiating the amplification reaction. As indicated in the table, an additional 2.4 cycles of reaction progress at the lowest input target level were required to achieve the same level of amplicon synthesis when using the primers where 5′ insert-binding sequence was reduced from 23 to 11 nucleotides in length. It is to be understood that this cycle number difference reflects a reduction of about 5.3 fold (or 2\hat{ }2.4) in the efficiency of initiating amplification. Clearly, hybridization of the primer sequence upstream of the joining region in the dual-specificity primer strongly influenced cooperative hybridization of the primer sequence downstream of the joining region. Thus, the results were consistent with a requirement for hybridization of both the upstream (5′ insert-hybridizing) and downstream (3′ target-hybridizing) sequences of the dual-specificity primer to initiate amplification reactions.

FIG. 6 illustrates at least three features of dual-specificity primers. First, the spacing-apart of linear plots in FIG. 6 reflected differences in the initial primer-template hybridization interaction preliminary to polymerase-dependent extension. More particularly, progressive shortening of 5′ (i.e., insert-binding) sequences in the different dual-specificity primers reduced the likelihood of an initial primer-template interaction, and so delayed the cooperative binding and extension of 3′ (i.e., target-binding) sequence of the primer. Shortening 5′ sequence of the dual-specificity primer below a length of 14 nucleotides (e.g., to 11 nucleotides) noticably compromised the initial primer hybridization and extension reaction, but had no impact on efficiency of amplification in subsequent amplification cycles. Second, each different dual-specificity primer amplified template nucleic acid with a similar efficiency, as judged by the linear relationships between input template concentrations and observed Tcycle values. This was because each different amplicon hybridized to the dual-specificity primer in its reaction mixture in the same fashion following the initial PCR cycle (see FIG. 1). Indeed, the duplex region between the dual-specificity primer and the amplicon was determined by the spacing of 3′ end of the target-binding sequence and the template termination moiety of the primer. Third, dual-specificity primers were highly tolerant of differences in spacing-apart of the two primer binding sites (e.g., 5′ insert-binding sequence, and 3′ target-binding sequence) in the template nucleic acid. Indeed, template nucleic acid amplified efficiently, regardless of whether the joining region was longer or shorter than the length of unpaired nucleotides in the hybridized template (see FIG. 5).

The showing that each of the different dual-specificity primers initiated amplification reactions from the MRSA template, but not from the MSSA template, confirmed utility of the dual-specificity primers for detecting two juxtaposed sequences with variable spacing therebetween. More particularly, hybrid duplexes between the cognate target nucleic acid and the dual-specificity primers exhibited different numbers of looped-out or non-hybridized bases. The duplex that included the primer of PRIMER 2 had one unpaired base in the template strand and nine unpaired bases in the joining region of the primer strand. The duplex that included the primer of PRIMER 4 had three unpaired bases in the template strand and nine unpaired bases in the joining region of the primer strand. The duplex that included the primer of PRIMER 5 had five unpaired bases in the template strand and nine unpaired bases in the joining region of the primer strand. The duplex that included the primer of PRIMER 6 had ten unpaired bases in the template strand and nine unpaired bases in the joining region of the primer strand. The duplex that included the primer of PRIMER 7 had thirteen unpaired bases in the template strand and nine unpaired bases in the joining region of the primer strand. Regardless of whether the looped-out region was longer in the primer strand or in the target nucleic acid strand (see FIG. 5), all primers initiated exponential amplification reactions in a template-specific manner. Once begun, amplification proceeded with similar efficiency in all cases as judged by the substantially parallel calibration plots in FIG. 6.

Taken together, the results demonstrated that dual-specificity primers were useful for amplifying target nucleic acid sequences adjacent to a junction, where upstream and downstream portions of the primer were complementary to first and second target nucleic acid sequences on opposite sides of the junction. Moreover, amplification reactions employing dual-specificity primers tolerated differences in the spacing between the two target nucleic acid sequences that were hybridized by a dual-specificity primer in the target nucleic acid. The results demonstrated that the number of nucleotides of the joining region of a dual-specificity primer could be the same as, greater than, or less than the number of nucleotides separating sequences in the first nucleic acid that are complementary to 5′ sequence of nucleotides and 3′ sequence of nucleotides of the dual-specificity primer. Accordingly, dual-specificity primers advantageously can amplify nucleic acid sequences adjacent to a junction between two sequences in a target nucleic acid without requiring precision in the positioning of the junction.

All of the compositions, kits, and methods disclosed and claimed herein can be made and executed without undue experimentation in light of the present disclosure. While the disclosure describes preferred embodiments, it will be apparent to those of skill in the art that variations may be applied without departing from the spirit and scope of the disclosure. All such variations and equivalents apparent to those skilled in the art, whether now existing or later developed, are deemed to be within the spirit and scope of the disclosure.

All patents, patent applications, and publications mentioned in the specification are indicative of the levels of those of ordinary skill in the art to which the disclosure pertains. All patents, patent applications, and publications are herein incorporated by reference in their entirety for all purposes and to the same extent as if each individual publication was specifically and individually indicated to be incorporated by reference in its entirety for any and all purposes.

1. A method of determining whether a test sample comprises a first nucleic acid without detecting a second nucleic acid, wherein the first and second nucleic acids comprise a common first sequence, and wherein the first and second nucleic acids have different sequences downstream from the common first sequence, the method comprising the steps of:
(a) contacting the test sample with a dual-specificity primer that comprises
(i) a 5′ sequence of nucleotides that stably hybridizes to the downstream sequence of the first nucleic acid without stably hybridizing to the downstream sequence of the second nucleic acid,
(ii) a 3′ sequence of nucleotides that is complementary to the common first sequence,
wherein 3′ sequence of nucleotides hybridizes to the common first sequence only when 5′ sequence of nucleotides is stably hybridized to the downstream sequence of the first nucleic acid,
(iii) a joining region between 5′ sequence of nucleotides and 3′ sequence of nucleotides, and
(iv) a template termination moiety positioned between 5′ sequence of nucleotides and the joining region,
wherein the joining region does not stably hybridize to any nucleic acid sequence that separates the common first sequence from either the downstream sequence of the first nucleic acid or the downstream sequence of the second nucleic acid;
(b) performing an amplification reaction,
wherein the amplification reaction comprises the dual-specificity primer and nucleic acids of the test sample;
(c) detecting formation of any of an amplicon comprising 3′ sequence of nucleotides or the complement thereof that may have been produced in the amplification reaction; and
(d) determining either that
(i) the test sample comprises the first nucleic acid if the amplicon is detected in step (c), or
(ii) the test sample does not comprise the first nucleic acid if the amplicon is not detected in step (c).

2. The method of claim 1, wherein step (a) comprises combining in a reaction vessel each of the test sample, the dual-specificity primer, a pH buffer, deoxyribonucleotide triphosphates (dNTPs), and at least one polymerase to form an amplification reaction mixture.

3. The method of claim 1, wherein each of steps (a) to (d) is carried out using an automated nucleic acid analyzer that comprises each of:
a temperature-regulated block or incubator that holds the reaction vessel and controls the temperature of the amplification reaction mixture therein,
a fluorometer in optical communication with the amplification reaction mixture contained in the reaction vessel, and
a computer that receives signals from the fluorometer.

4. (canceled)

5. (canceled)

6. The method of claim 1, wherein step (c) comprises detecting with an optical sensor.

7. (canceled)

8. The method of claim 1, wherein the amplification reaction in step (b) comprises a PCR reaction, and wherein the amplicon in step (c) comprises DNA.

9. (canceled)

10. The method of claim 1, wherein step (c) comprises detecting as the amplification reaction is occurring, and further comprises determining a threshold cycle value indicating a predetermined level of progress in the amplification reaction.

11. (canceled)

12. The method of claim 1, wherein step (c) comprises detecting with a hybridization probe, wherein the hybridization probe comprises a detectable label.

13. (canceled)

14. The method of claim 1, wherein the template termination moiety is positioned between 5′ sequence of nucleotides and the joining region to fully separate one from the other, and wherein the template termination moiety of the dual-specificity primer comprises a non-nucleotide moiety.

15. The method of claim 14, wherein the non-nucleotide moiety comprises at least one glycol linkage.

16. The method of claim 1, wherein;
(a) 5′ sequence of nucleotides of the dual-specificity primer is 10 to 60 nucleotides in length;
(b) 3′ sequence of nucleotides of the dual-specificity primer is 6 to 10 nucleotides in length; or
(c) the joining region of the dual-specificity primer is 5 to 50 nucleotides in length.

17. (canceled)

18. (canceled)

19. The method of claim 1, wherein the joining region of the dual specificity primer is 5 to 50 nucleotides in length and:
(a) the joining region of the dual-specificity primer is of a length that is the same as the number of nucleotides separating sequences in the first nucleic acid that are complementary to the 5′ sequence of nucleotides and 3′ sequence of nucleotides of the dual-specificity primer; or
(b) the joining region of the dual-specificity primer is of a length that is greater than the number of nucleotides separating sequences in the first nucleic acid that are complementary to the 5′ sequence of nucleotides and 3′ sequence of nucleotides of the dual-specificity primer; or
(c) the joining region of the dual-specificity primer is of a length that is less than the number of nucleotides separating sequences in the first nucleic acid that are complementary to the 5′ sequence of nucleotides and 3′ sequence of nucleotides of the dual-specificity primer.

20. (canceled)

21. (canceled)

22. The method of claim 1, wherein 5′ sequence of nucleotides and 3′ sequence of nucleotides have a combined length of 17 to 29 nucleotides.

23. The method of claim 22, wherein 5′ sequence of nucleotides is 11 to 23 nucleotides long.

24. The method of claim 2, wherein the amplification reaction mixture further comprises an amplification oligonucleotide that hybridizes to an extension product of the dual-specificity primer when using the first nucleic acid as a template.

25. The method of claim 24, wherein the amplification oligonucleotide is a primer having a 3′ hydroxyl moiety, and wherein the primer having the 3′ hydroxyl moiety is not a dual-specificity primer.

26. The method of claim 1, wherein step (d) is automated by a computer programmed with software.

27. The method of claim 1, wherein the amplification reaction further comprises an internal control nucleic acid that amplifies to produce an internal control amplicon even in the absence of the first or second nucleic acids.

28. The method of claim 1, wherein determining in step (d) that the test sample comprises the first nucleic acid is an indication that either a chromosomal rearrangement or a post-transcriptional RNA processing event occurred to join together the common first sequence and the downstream sequence of the first nucleic acid.

29. (canceled)

30. (canceled)

31. A dual-specificity primer for amplifying a first nucleic acid without amplifying a second nucleic acid in the same nucleic acid amplification reaction, wherein the first and second nucleic acids comprise a common first sequence, and wherein the first and second nucleic acids have different sequences downstream from the common first sequence, the dual-specificity primer comprising:
(i) a 5′ sequence of nucleotides that stably hybridizes to the downstream sequence of the first nucleic acid without stably hybridizing to the downstream sequence of the second nucleic acid,
(ii) a 3′ sequence of nucleotides that is complementary to the common first sequence,
wherein 3′ sequence of nucleotides hybridizes to the common first sequence only when 5′ sequence of nucleotides is stably hybridized to the downstream sequence of the first nucleic acid,
(iii) a joining region between 5′ sequence of nucleotides and 3′ sequence of nucleotides, and
(iv) a template termination moiety positioned between 5′ sequence of nucleotides and the joining region,
wherein the joining region does not stably hybridize to any nucleic acid sequence that separates the common first sequence from either the downstream sequence of the first nucleic acid or the downstream sequence of the second nucleic acid.

32-44. (canceled)

45. A kit containing one or more reagents for amplifying a first nucleic acid without amplifying a second nucleic acid in the same nucleic acid amplification reaction, wherein the first and second nucleic acids comprise a common first sequence, and wherein the first and second nucleic acids have different sequences downstream from the common first sequence, the kit comprising, in one or more vials:
a dual-specificity primer that comprises
(i) a 5′ sequence of nucleotides that stably hybridizes to the downstream sequence of the first nucleic acid without stably hybridizing to the downstream sequence of the second nucleic acid,
(ii) a 3′ sequence of nucleotides that is complementary to the common first sequence,
wherein 3′ sequence of nucleotides hybridizes to the common first sequence only when 5′ sequence of nucleotides is stably hybridized to the downstream sequence of the first nucleic acid,
(iii) a joining region between 5′ sequence of nucleotides and 3′ sequence of nucleotides, and
(iv) a template termination moiety positioned between 5′ sequence of nucleotides and the joining region,
wherein the joining region does not stably hybridize to any nucleic acid sequence that separates the common first sequence from either the downstream sequence of the first nucleic acid or the downstream sequence of the second nucleic acid; and
an amplification oligomer, the amplification oligomer being substantially complementary to a polymerase-dependent extension product of the dual-specificity primer using the first nucleic acid as a template,
wherein the dual-specificity primer and the amplification oligomer are configured to produce a first nucleic acid amplicon when used in combination with the first nucleic acid as the template in a nucleic acid amplification reaction.

46-67. (canceled)


SEQUENCE LISTING (15 sequences)

SEQ ID NO: 1 - DNA, 18 bp, Staphylococcus aureus
  source 1..18: mol_type = unassigned DNA
  misc_feature 1..18: function = Reverse primer
ccctttatgaagcggctg

SEQ ID NO: 2 - DNA, 23 bp, Staphylococcus aureus
  source 1..23: mol_type = unassigned DNA
ccgcatcatttatgatatgcttc

SEQ ID NO: 3 - DNA, 23 bp, Staphylococcus aureus
  source 1..23: mol_type = unassigned DNA
ccgcatcatttatgatatgcttc

SEQ ID NO: 4 - DNA, 21 bp, Staphylococcus aureus
  source 1..21: mol_type = unassigned DNA
ccgcatcatttatgatatgct

SEQ ID NO: 5 - DNA, 19 bp, Staphylococcus aureus
  source 1..19: mol_type = unassigned DNA
ccgcatcatttatgatatg

SEQ ID NO: 6 - DNA, 14 bp, Staphylococcus aureus
  source 1..14: mol_type = unassigned DNA
ccgcatcatttatg

SEQ ID NO: 7 - DNA, 11 bp, Staphylococcus aureus
  source 1..11: mol_type = unassigned DNA
ccgcatcattt

SEQ ID NO: 8 - DNA, 17 bp, Staphylococcus aureus
  source 1..17: mol_type = unassigned DNA
  misc_feature 1..17: function = Forward primer
gattggcccaagaattg

SEQ ID NO: 9 - DNA, 24 bp, Staphylococcus aureus
  source 1..24: mol_type = unassigned DNA
  misc_feature 24: function = Hexanediol attached at 3' position of nucleotide 24
  misc_feature 1..24: function = Primary Invader probe
cgcgccgaggaaccaacgcatgac

SEQ ID NO: 10 - DNA, 33 bp, synthetic construct
  source 1..33: mol_type = other DNA
  misc_feature 1: function = Fluorescein attached at nucleotide position 1
  misc_feature 1..33: function = FRET cassette
  misc_feature 4: function = Quencher moiety attached at nucleotide position 4
  misc_feature 33: function = Hexane diol attached at 3' position of nucleotide 33
tctagccggttttccggctgagacctcggcgcg

SEQ ID NO: 11 - DNA, 62 bp, Staphylococcus aureus
  source 1..62: mol_type = genomic DNA
  misc_feature 1..62: function = Sequence representing MSSA
agattatgcgaggagaggcgtatcataagtaaaactaaaaaattctgtatgaggagataata

SEQ ID NO: 12 - DNA, 62 bp, Staphylococcus aureus
  source 1..62: mol_type = genomic DNA
  misc_feature 1..62: function = Sequence representing MRSA
agattatgcgtggagaagcatatcataaatgatgcggttttttcagccgcttcataaaggga

SEQ ID NO: 13 - DNA, 55 bp, Staphylococcus aureus
  source 1..55: mol_type = genomic DNA
  misc_feature 1..55: function = Partial MRSA sequence
atagagcatttaagattatgcgtggagaagcatatcataaatgatgcggtttttt

SEQ ID NO: 14 - DNA, 15 bp, Staphylococcus aureus
  source 1..15: mol_type = unassigned DNA
gtcatcaggccacgc

SEQ ID NO: 15 - DNA, 18 bp, Staphylococcus aureus
  source 1..18: mol_type = unassigned DNA
gtcatcaggccacgcata